(19) United States
(12) Patent Application Publication
(10) Pub. No.: US 2004/0205847 A1
(43) Pub. Date: Oct. 14, 2004

(54) GDC-1 genes conferring herbicide resistance

(75) Inventors: Philip E. Hammer, Cary, NC (US); Todd K. Hinson, Rougemont, NC (US); Brian Carr, Raleigh, NC (US); Nicholas B. Duck, Apex, NC (US)

Correspondence Address: ALSTON & BIRD LLP, BANK OF AMERICA PLAZA, 101 SOUTH TRYON STREET, SUITE 4000, CHARLOTTE, NC, 28280-4000, US

(73) Assignee: Athenix Corporation, Durham, NC (US)

(21) Appl. No.: 10/796,953

(22) Filed: Mar. 10, 2004

Related U.S. Application Data
(60) Provisional application No. 60/453,237, filed on Mar. 10, 2003.

(51) Int. Cl. A01H 1/00; C12N 15/82; C07H 21/04; C07K 14/435

(52) U.S. Cl. 800/288; 435/69.1; 435/320.1; 435/468; 435/419; 530/350; 536/23.5

(57) ABSTRACT

Compositions and methods for conferring herbicide resistance to plants, plant cells, tissues and seeds are provided. Compositions comprising a coding sequence for a polypeptide that confers resistance or tolerance to glyphosate herbicides are provided. The coding sequences can be used in DNA constructs or expression cassettes for transformation and expression in plants. Compositions also comprise transformed plants, plant cells, tissues, and seeds. In particular, isolated nucleic acid molecules corresponding to glyphosate resistant nucleic acid sequences are provided. Additionally, amino acid sequences corresponding to the polynucleotides are encompassed. In particular, the present invention provides for isolated nucleic acid molecules comprising nucleotide sequences encoding an amino acid sequence shown in SEQ ID NO:3, 6, 8, 11, 19, or 21, or a nucleotide sequence set forth in SEQ ID NO:1, 2, 4, 5, 7, 9, 10, 18, or 20, as well as variants and fragments thereof.

### CROSS-REFERENCE TO RELATED APPLICATIONS

The present application claims the benefit of U.S. Provisional Application Serial No. 60/453, 237, filed Mar. 10, 2003, the contents of which are herein incorporated by reference in their entirety.

### FIELD OF THE INVENTION

This invention provides novel genes encoding herbicide resistance, which are useful in plant biology, crop breeding, and plant cell culture.

### BACKGROUND OF THE INVENTION

N-phosphonomethylglycine, commonly referred to as glyphosate, is an important agronomic chemical. Glyphosate inhibits the enzyme that converts phosphoenolpyruvic acid (PEP) and 3-phosphoshikimic acid to 5-enolpyruvyl-3-phosphoshikimic acid. Inhibition of this enzyme (5-enolpyruvylshikimate-3-phosphate synthase; referred to herein as “EPSP synthase”) kills plant cells by shutting down the shikimate pathway, thereby inhibiting aromatic acid biosynthesis.

Since glyphosate-class herbicides inhibit aromatic amino acid biosynthesis, they not only kill plant cells, but are also toxic to bacterial cells. Glyphosate inhibits many bacterial EPSP synthases, and thus is toxic to these bacteria. However, certain bacterial EPSP synthases may have a high tolerance to glyphosate.

Plant cells resistant to glyphosate toxicity can be produced by transforming plant cells to express glyphosate-resistant EPSP synthases. A mutated EPSP synthase from Salmonella typhimurium strain CT7 confers glyphosate resistance in bacterial cells, and confers glyphosate resistance on plant cells (U.S. Pat. Nos. 4,535,060, 4,769,061, and 5,094,945). Thus, there is a precedent for use of glyphosate-resistant bacterial EPSP synthases to confer glyphosate resistance upon plant cells.

An alternative method to generate target genes resistant to a toxin (such as an herbicide) is to identify and develop enzymes that result in detoxification of the toxin to an inactive or less active form. This can be accomplished by identifying enzymes that encode resistance to the toxin in a toxin-sensitive test organism, such as a bacterium.

Castle et al (WO 02/36782 A2) describe proteins (glyphosate N-acetyltransferases) that are described as modifying glyphosate by acetylation of a secondary amine to yield N-acetylglyphosate.

Barry et al (U.S. Pat. No. 5,463,175) describes genes encoding an oxidoreductase (GOX), and states that GOX proteins degrade glyphosate by removing the phosphonate residue to yield amino methyl phosphonic acid (AMPA). This suggests that glyphosate resistance can also be conferred, at least partially, by removal of the phosphonate group from glyphosate. However, the resulting compound (AMPA) appears to provide reduced but measurable toxicity upon plant cells. Barry describes the effect of AMPA accumulation on plant cells as resulting in effects including chlorosis of leaves, infertility, stunted growth, and death. Barry (U.S. Pat. No. 6,448,476) describes plant cells expressing an AMPA-N-acetyltransferase (phnO) to detoxify AMPA.

Phophonates, such as glyphosate, can also be degraded by cleavage of C-P bond by a C-P lyase. Wacket et al. (1987) J. Bacteriol. 169:710-717 described strains that utilize glyphosate as a sole phosphate source. Kishore et al. (1987) J. Biol. Chem. 262:12164-12168 and Shinabarger et al. (1986) J. Bacteriol. 168:702-707 describe degradation of glyphosate by C-P Lyase to yield glycine and inorganic phosphate.

While several strategies are available for detoxification of toxins, such as the herbicide glyphosate, as described above, new activities capable of degrading glyphosate are useful. Novel genes and genes conferring glyphosate resistance by novel mechanisms of action would be of additional usefulness. Single genes conferring glyphosate resistance by formation of non-toxic products would be especially useful.

Thus, novel genes encoding resistance to herbicides are needed.

### SUMMARY OF INVENTION

Compositions and methods for conferring herbicide resistance to plants, plant cells, tissues and seeds are provided. Compositions comprising a coding sequence for a polypeptide that confers resistance or tolerance to glyphosate herbicides are provided. The coding sequences can be used in DNA constructs or expression cassettes for transformation and expression in plants. Compositions also comprise transformed plants, plant cells, tissues, and seeds.

In particular, isolated nucleic acid molecules corresponding to glyphosate resistance-conferring nucleic acid sequences are provided. Additionally, amino acid sequences corresponding to the polynucleotides are encompassed. In particular, the present invention provides for isolated nucleic acid molecules comprising nucleotide sequences encoding an amino acid sequence shown in SEQ ID NO:3, 6, 8, 11, 19, or 21, or a nucleotide sequence set forth in SEQ ID NO:1, 2, 4, 5, 7, 9, 10, 18, or 20, as well as variants and fragments thereof. Nucleotide sequences that are complementary to a nucleotide sequence of the invention, or that hybridize to a sequence of the invention are also encompassed.

### DESCRIPTION OF FIGURES

FIG. 1 is a diagram that shows GDC-1 (full), GDC-1 (23), GDC-1 (35), GDC-1 (59), and GDC-1 (35 H3mut), as well as the location of the TPP binding domains and the location (X) of a mutation.

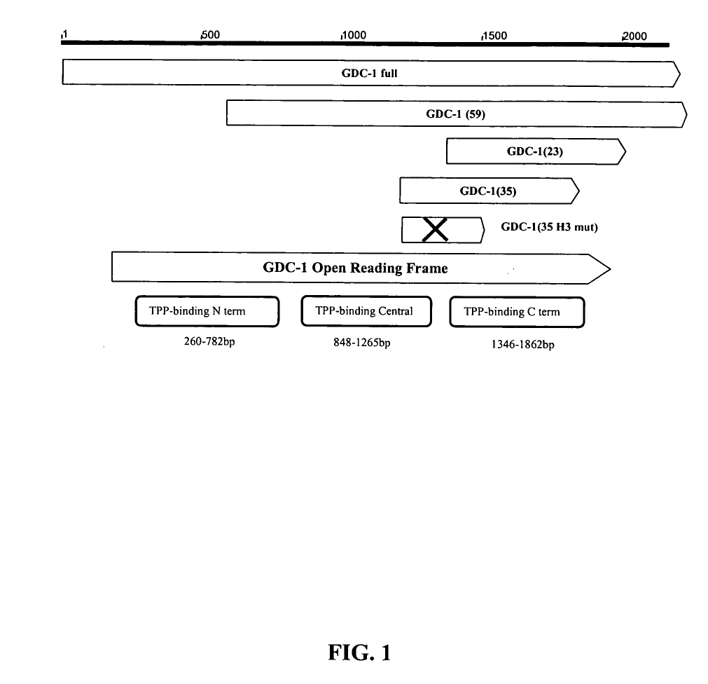

FIG. 2 shows an alignment of the predicted proteins resulting from translation of the clones GDC-1 (full) (SEQ ID NO:19), GDC-1 (23) (SEQ ID NO:6), GDC-1 (35) (SEQ ID NO:8), and GDC-1 (59) (SEQ ID NO:11).

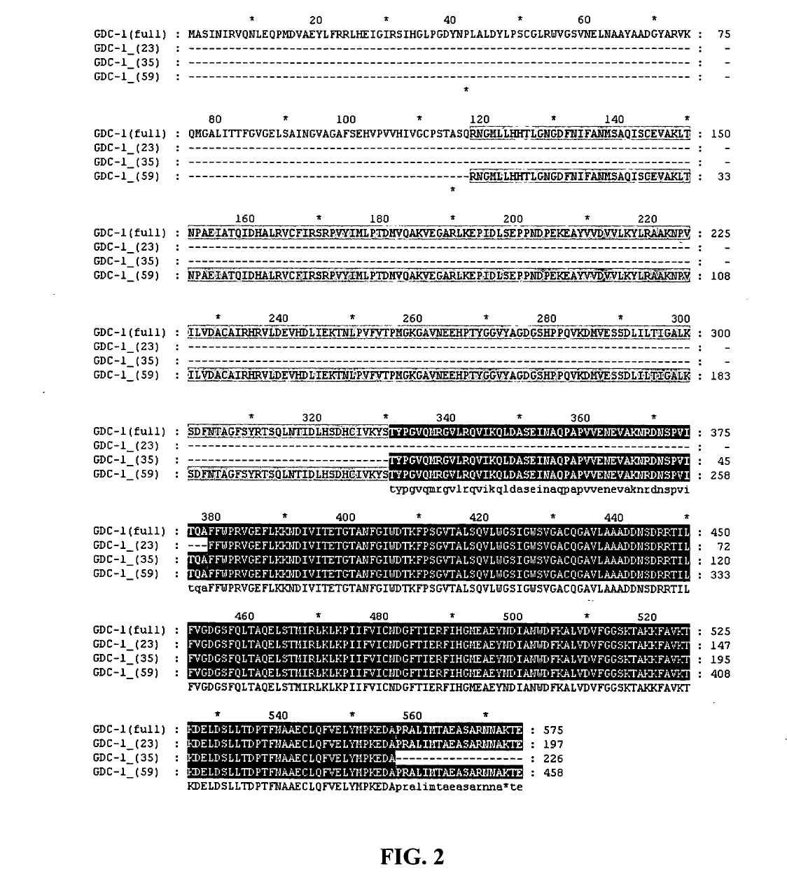

FIG. 3 shows an alignment of GDC-1 protein (SEQ ID NO:19) to pyruvate decarboxylase of Saccharomyces cerevesiae (SEQ ID NO:13), a putative indole-3-pyruvate decarboxylase from Salmonella typhimurium (SEQ ID NO:14), pyruvate decarboxylase (EC 4.1.1.1) from Zymomonas mobilis (SEQ ID NO:15), acetolactate synthase from Saccharomyces cerevesiae (SEQ ID NO:16), and acetolactate synthase from Magnaporthe grisea (SEQ ID NO:17). The alignment shows the most highly conserved amino acid residues highlighted in black, and highly conserved amino acid residues highlighted in gray.

FIG. 4 shows the growth of GDC-1 expressing cells at various concentrations of glyphosate as compared to vector and media only controls at 42 hours. Growth was measured by absorbance at 600 nm.

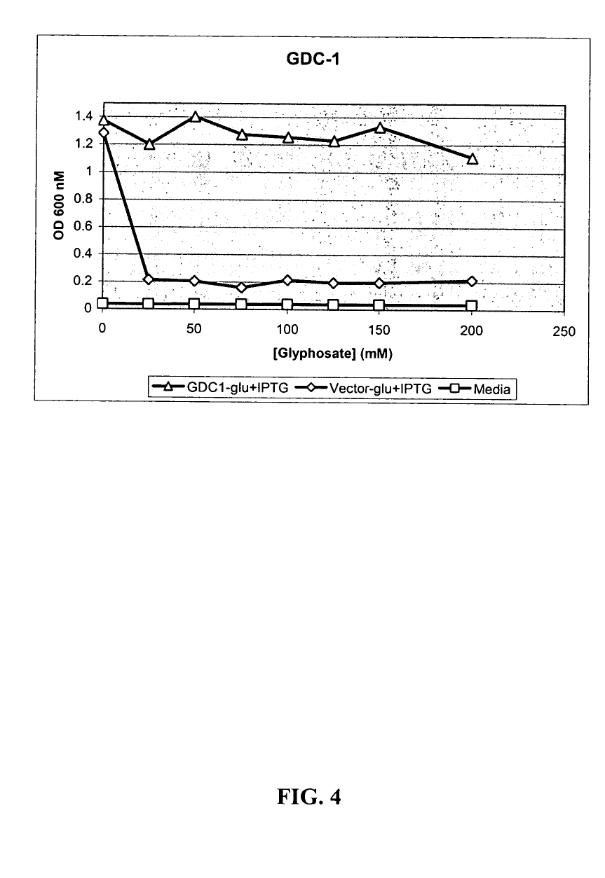

FIGS. 5A shows the HPLC column elution profile of C¹⁴ from a sample not incubated with GDC-1, and FIG. 5B shows the HPLC column elution profile of C¹⁴ after incubation with 100 ng GDC-1.

### DETAILED DESCRIPTION

The present invention is drawn to compositions and methods for regulating resistance in organisms, particularly in plants or plant cells. The methods involve transforming organisms with nucleotide sequences encoding a glyphosate resistance protein of the invention. In particular, the nucleotide sequences of the invention are useful for preparing plants that show increased tolerance to the herbicide glyphosate. Thus, transformed plants, plant cells, plant tissues and seeds are provided. Compositions include nucleic acids and proteins relating to glyphosate tolerance in plants as well as transformed plants, plant tissues and seeds. More particularly, nucleotide sequences encoding all or part of the “glyphosate resistance-conferring decarboxylase” gene GDC-1 and the amino acid sequences of the proteins encoded thereby are disclosed. The sequences find use in the construction of expression vectors for subsequent transformation into organisms of interest, as probes for the isolation of other glyphosate resistance genes, as selectable markers, and the like.

### Definitions

“Glyphosate” includes any herbicidal form of N-phosphonomethylglycine (including any salt thereof) and other forms that result in the production of the glyphosate anion in planta.

“Glyphosate (or herbicide) resistance-conferring decarboxylase” or “GDC” includes a DNA segment that encodes all or part of a glyphosate (or herbicide) resistance protein. This includes DNA segments that are capable of expressing a protein that confers glyphosate (herbicide) resistance to a cell.

An “herbicide resistance protein” or a protein resulting from expression of an “herbicide resistance-encoding nucleic acid molecule” includes proteins that confer upon a cell the ability to tolerate a higher concentration of an herbicide than cells that do not express the protein, or to tolerate a certain concentration of an herbicide for a longer time than cells that do not express the protein.

A “glyphosate resistance protein” includes a protein that confers upon a cell the ability to tolerate a higher concentration of glyphosate than cells that do not express the protein, or to tolerate a certain concentration of glyphosate for a longer time than cells that do not express the protein. By “tolerate” or “tolerance” is intended either to survive, or to carry out essential cellular functions, such as protein synthesis and respiration, in a manner that is not readily discemable from untreated cells.

By “decarboxylase” is intended a protein, or a gene encoding a protein, whose catalytic mechanism can include cleavage and release of a carboxylic acid. This includes enzymes that liberate CO₂, such as pyruvate decarboxlyases, acetolactate synthases, and orthinine decarboxylases, as well as enzymes that liberate larger carboxylic acids. “Decarboxylase” includes proteins that utilize thiamine pyrophoshate as a cofactor in enzymatic catalysis. Many such decarbolyases also utilize other cofactors, such as FAD.

By “TPP-binding domain” is intended a region of conserved amino acids present in enzymes that are capable of utilizing TPP as a cofactor.

“Plant tissue” includes all known forms of plants, including undifferentiated tissue (e.g. callus), suspension culture cells, protoplasts, plant cells including leaf cells, root cells and phloem cells, plant seeds, pollen, propagules, embryos and the like.

“Plant expression cassette” includes DNA constructs that are capable of resulting in the expression of a protein from an open reading frame in a plant cell. Typically these contain a promoter and a coding sequence. Often, such constructs will also contain a 3′ untranslated region. Such constructs may contain a ‘signal sequence’ or ‘leader sequence’ to facilitate co-translational or post-translational transport of the peptide to certain intracellular structures such as the chloroplast (or other plastid), endoplasmic reticulum, or Golgi apparatus.

“Signal sequence” includes sequences that are known or suspected to result in cotranslational or post-translational peptide transport across the cell membrane. In eukaryotes, this typically involves secretion into the Golgi apparatus, with some resulting glycosylation.

“Leader sequence” includes any sequence that when translated, results in an amino acid sequence sufficient to trigger co-translational transport of the peptide chain to a sub-cellular organelle. Thus, this includes leader sequences targeting transport and/or glycosylation by passage into the endoplasmic reticulum, passage to vacuoles, plastids including chloroplasts, mitochondria, and the like.

“Plant transformation vector” includes DNA molecules that are necessary for efficient transformation of a plant cell. Such a molecule may consist of one or more plant expression cassettes, and may be organized into more than one ‘vector’ DNA molecule. For example, binary vectors are plant transformation vectors that utilize two non-contiguous DNA vectors to encode all requisite cis- and trans-acting functions for transformation of plant cells (Hellens and Mullineaux (2000) Trends in Plant Science 5:446-451).

“Vector” refers to a nucleic acid construct designed for transfer between different host cells. “Expression vector” refers to a vector that has the ability to incorporate, integrate and express heterologous DNA sequences or fragments in a foreign cell.

“Transgenic plants” or “transformed plants” or “stably transformed plants or cells or tissues” refers to plants that have incorporated or integrated exogenous or endogenous nucleic acid sequences or DNA fragments or chimeric nucleic acid sequences or fragments.

“Heterologous” generally refers to the nucleic acid sequences that are not endogenous to the cell or part of the native genome in which they are present, and have been added to the cell by infection, transfection, microinjection, electroporation, microprojection, or the like.

“Promoter” refers to a nucleic acid sequence that functions to direct transcription of a downstream coding sequence. The promoter together with other transcriptional and translational regulatory nucleic acid sequences (also termed “control sequences”) are necessary for the expression of a DNA sequence of interest.

Provided here is a novel isolated gene that confers resistance to glyphosate. Also provided are amino acid sequences of the GDC-1 protein. The protein resulting from translation of this gene allows cells to function in the presence of concentrations of glyphosate that are otherwise toxic to cells, including plant cells and bacterial cells.

An “isolated” or “purified” nucleic acid molecule or protein, or biologically active portion thereof, is substantially free of other cellular material, or culture medium when produced by recombinant techniques, or substantially free of chemical precursors or other chemicals when chemically synthesized. Preferably, an “isolated” nucleic acid is free of sequences (preferably protein encoding sequences) that naturally flank the nucleic acid (i.e., sequences located at the 5′ and 3′ ends of the nucleic acid) in the genomic DNA of the organism from which the nucleic acid is derived. For purposes of the invention, “isolated” when used to refer to nucleic acid molecules excludes isolated chromosomes. For example, in various embodiments, the isolated glyphosate resistance-encoding nucleic acid molecule can contain less than about 5 kb, 4 kb, 3 kb, 2 kb, 1 kb, 0.5 kb, or 0.1 kb of nucleotide sequence that naturally flanks the nucleic acid molecule in genomic DNA of the cell from which the nucleic acid is derived. A glyphosate resistance protein that is substantially free of cellular material includes preparations of protein having less than about 30%, 20%, 10%, or 5% (by dry weight) of non-glyphosate resistance protein (also referred to herein as a “contaminating protein”). Various aspects of the invention are described in further detail in the following subsections.

### Isolated Nucleic Acid Molecules, and Variants and Fragments Thereof

One aspect of the invention pertains to isolated nucleic acid molecules comprising nucleotide sequences encoding glyphosate resistance proteins and polypeptides or biologically active portions thereof, as well as nucleic acid molecules sufficient for use as hybridization probes to identify glyphosate resistance-encoding nucleic acids. As used herein, the term “nucleic acid molecule” is intended to include DNA molecules (e.g., cDNA or genomic DNA) and RNA molecules (e.g., mRNA) and analogs of the DNA or RNA generated using nucleotide analogs. The nucleic acid molecule can be single-stranded or double-stranded, but preferably is double-stranded DNA.

Nucleotide sequences encoding the proteins of the present invention include the sequences set forth in SEQ ID NOS:1, 2, 18, and 20, and complements thereof. By “complement” is intended a nucleotide sequence that is sufficiently complementary to a given nucleotide sequence such that it can hybridize to the given nucleotide sequence to thereby form a stable duplex. The corresponding amino acid sequences for the glyphosate resistance proteins encoded by the nucleotide sequences are set forth in SEQ ID NOS:3, 19, and 21. The invention also encompasses nucleic acid molecules comprising nucleotide sequences encoding partial-length glyphosate resistance proteins, including the sequences set forth in SEQ ID NOS:4, 5, 7, 9, and 10, and complements thereof. The corresponding amino acid sequences for the glyphosate resistance proteins encoded by these partial-length nucleotide sequences are set forth in SEQ ID NOS:6, 8, and 11.

Nucleic acid molecules that are fragments of these glyphosate resistance-encoding nucleotide sequences are also encompassed by the present invention. By “fragment” is intended a portion of the nucleotide sequence encoding a glyphosate resistance protein. A fragment of a nucleotide sequence may encode a biologically active portion of a glyphosate resistance protein, or it may be a fragment that can be used as a hybridization probe or PCR primer using methods disclosed below. Nucleic acid molecules that are fragments of a glyphosate resistance nucleotide sequence comprise at least about 15, 20, 50, 75, 100, 200, 300, 350, 400, 450, 500, 550, 600, 650, 700, 750, 800, 850, 900, 950, 1000, 1050, 1100, 1150, 1200, 1250, 1300, 1350, 1400, 1450, 1500, 1550, 1600, 1650, 1700, 1750, 1800, 1850, 1900, 1950, 2000, 2050, 2100, 2150, 2200 nucleotides, or up to the number of nucleotides present in a full-length glyphosate resistance-encoding nucleotide sequence disclosed herein (for example, 2210 nucleotides for SEQ ID NO:1) depending upon the intended use.

Fragments of the nucleotide sequences of the present invention generally will encode protein fragments that retain the biological activity of the full-length glyphosate resistance protein; i.e., glyphosate resistance activity. By “retains glyphosate resistance activity” is intended that the fragment will have at least about 30%, preferably at least about 50%, more preferably at least about 70%, even more preferably at least about 80% of the glyphosate resistance activity of the full-length glyphosate resistance protein disclosed herein as SEQ ID NO:19. Methods for measuring glyphosate resistance activity are well known in the art. See, for example, U.S. Pat. Nos. 4,535,060, and 5,188,642, each of which are herein incorporated by reference in their entirety.

A fragment of a glyphosate resistance-encoding nucleotide sequence that encodes a biologically active portion of a protein of the invention will encode at least about 15, 25, 30, 50, 75, 100, 125, 150, 175, 200, 250, 300, 350, 400, 450, 500, or 550 contiguous amino acids, or up to the total number of amino acids present in a full-length glyphosate resistance protein of the invention (for example, 575 amino acids for SEQ ID NO:3).

Preferred glyphosate resistance proteins of the present invention are encoded by a nucleotide sequence sufficiently identical to the nucleotide sequence of SEQ ID NO:1, 2, 4, 5, 7, 9, 10, 18, or 20. The term “sufficiently identical is intended an amino acid or nucleotide sequence that has at least about 60% or 65% sequence identity, preferably about 70% or 75% sequence identity, more preferably about 80% or 85% sequence identity, most preferably about 90%, 91%, 92%, 93%, 94%, 95%, 96%, 97%, 98% or 99% sequence identity compared to a reference sequence using one of the alignment programs described herein using standard parameters. One of skill in the art will recognize that these values can be appropriately adjusted to determine corresponding identity of proteins encoded by two nucleotide sequences by taking into account codon degeneracy, amino acid similarity, reading frame positioning, and the like.

To determine the percent identity of two amino acid sequences or of two nucleic acids, the sequences are aligned for optimal comparison purposes. The percent identity between the two sequences is a function of the number of identical positions shared by the sequences (i.e., percent identity=number of identical positions/total number of positions (e.g., overlapping positions)×100). In one embodiment, the two sequences are the same length. The percent identity between two sequences can be determined using techniques similar to those described below, with or without allowing gaps. In calculating percent identity, typically exact matches are counted.

The determination of percent identity between two sequences can be accomplished using a mathematical algorithm. A nonlimiting example of a mathematical algorithm utilized for the comparison of two sequences is the algorithm of Karlin and Altschul (1990) Proc. Natl. Acad. Sci. USA 87:2264, modified as in Karlin and Altschul (1993) Proc. Natl. Acad. Sci. USA 90:5873-5877. Such an algorithm is incorporated into the BLASTN and BLASTX programs of Altschul et al. (1990) J. Mol. Biol. 215:403. BLAST nucleotide searches can be performed with the BLASTN program, score=100, wordlength=12, to obtain nucleotide sequences homologous to GDC-like nucleic acid molecules of the invention. BLAST protein searches can be performed with the BLASTX program, score=50, wordlength=3, to obtain amino acid sequences homologous to glyphosate resistance protein molecules of the invention. To obtain gapped alignments for comparison purposes, Gapped BLAST can be utilized as described in Altschul et al. (1997) Nucleic Acids Res. 25:3389. Alternatively, PSI-Blast can be used to perform an iterated search that detects distant relationships between molecules. See Altschul et al. (1997) supra. When utilizing BLAST, Gapped BLAST, and PSI-Blast programs, the default parameters of the respective programs (e.g., BLASTX and BLASTN) can be used. See www.ncbi.nlm.nih.gov. Another non-limiting example of a mathematical algorithm utilized for the comparison of sequences is the ClustalW algorithm (Higgins et al. (1994) Nucleic Acids Res. 22:4673-4680). ClustalW compares sequences and aligns the entirety of the amino acid or DNA sequence, and thus can provide data about the sequence conservation of the entire amino acid sequence. The ClustalW algorithm is used in several commercially available DNA/amino acid analysis software packages, such as the ALIGNX module of the vector NTi Program Suite (Informax, Inc). After alignment of amino acid sequences with ClustalW, the percent amino acid identity can be assessed. A non-limiting example of a software program useful for analysis of ClustalW alignments is GeneDoc™. Genedoc™ (Karl Nicholas) allows assessment of amino acid (or DNA) similarity and identity between multiple proteins. Another non-limiting example of a mathematical algorithm utilized for the comparison of sequences is the algorithm of Myers and Miller (1988) CABIOS 4:11-17. Such an algorithm is incorporated into the ALIGN program (version 2.0), which is part of the GCG sequence alignment software package (available from Accelrys, Inc., 9865 Scranton Rd., San Diego, Calif., USA). When utilizing the ALIGN program for comparing amino acid sequences, a PAM120 weight residue table, a gap length penalty of 12, and a gap penalty of 4 can be used.

A preferred program is GAP version 10, which used the algorithm of Needleman and Wunsch (1970) supra. GAP Version 10 may be used with the following parameters: % identity and % similarity for a nucleotide sequence using GAP Weight of 50 and Length Weight of 3, and the nwsgapdna.cmp scoring matrix; % identity and % similarity for an amino acid sequence using GAP Weight of 8 and Length Weight of 2, and the BLOSUM62 Scoring Matrix. Equivalent programs may also be used. By “equivalent program” is intended any sequence comparison program that, for any two sequences in question, generates an alignment having identical nucleotide or amino acid residue matches and an identical percent sequence identity when compared to the corresponding alignment generated by GAP Version 10.

The invention also encompasses variant nucleic acid molecules. “Variants” of the glyphosate resistance-encoding nucleotide sequences include those sequences that encode the glyphosate resistance proteins disclosed herein but that differ conservatively because of the degeneracy of the genetic code, as well as those that are sufficiently identical as discussed above. Naturally occurring allelic variants can be identified with the use of well-known molecular biology techniques, such as polymerase chain reaction (PCR) and hybridization techniques as outlined below. Variant nucleotide sequences also include synthetically derived nucleotide sequences that have been generated, for example, by using site-directed mutagenesis but which still encode the glyphosate resistance proteins disclosed in the present invention as discussed below. Variant proteins encompassed by the present invention are biologically active, that is they retain the desired biological activity of the native protein, that is, glyphosate resistance activity. By “retains glyphosate resistance activity” is intended that the variant will have at least about 30%, preferably at least about 50%, more preferably at least about 70%, even more preferably at least about 80% of the glyphosate resistance activity of the native protein. Methods for measuring glyphosate resistance activity are well known in the art. See, for example, U.S. Pat. Nos. 4,535,060, and 5,188,642, each of which are herein incorporated by reference in their entirety.

The skilled artisan will further appreciate that changes can be introduced by mutation into the nucleotide sequences of the invention thereby leading to changes in the amino acid sequence of the encoded glyphosate resistance proteins, without altering the biological activity of the proteins. Thus, variant isolated nucleic acid molecules can be created by introducing one or more nucleotide substitutions, additions, or deletions into the corresponding nucleotide sequence disclosed herein, such that one or more amino acid substitutions, additions or deletions are introduced into the encoded protein. Mutations can be introduced by standard techniques, such as site-directed mutagenesis and PCR-mediated mutagenesis. Such variant nucleotide sequences are also encompassed by the present invention.

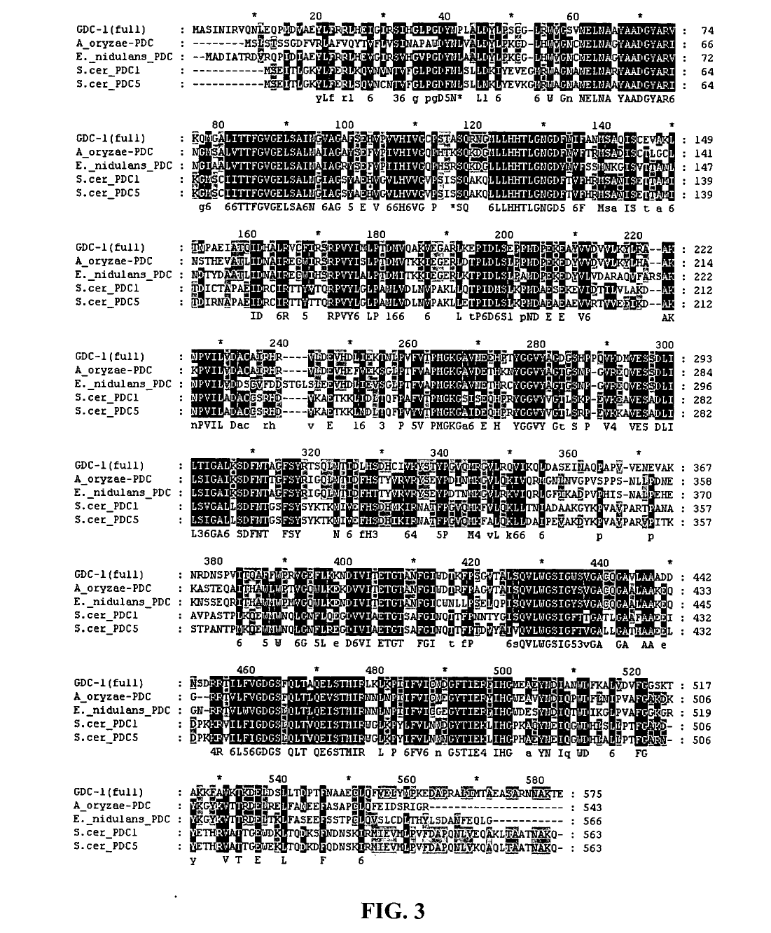

For example, conservative amino acid substitutions may be made at one or more predicted, preferably nonessential amino acid residues. A “nonessential” amino acid residue is a residue that can be altered from the wild-type sequence of a glyphosate resistance protein without altering the biological activity, whereas an “essential” amino acid residue is required for biological activity. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Amino acid substitutions may be made in nonconserved regions that retain function. In general, such substitutions would not be made for conserved amino acid residues, or for amino acid residues residing within a conserved motif, where such residues are essential for protein activity. Examples of residues that are conserved and that may be essential for protein activity include, for example, residues that are identical between all proteins contained in the alignment of FIG. 3. However, one of skill in the art would understand that functional variants may have minor conserved or nonconserved alterations in the conserved residues.

Alternatively, variant nucleotide sequences can be made by introducing mutations randomly along all or part of the coding sequence, such as by saturation mutagenesis, and the resultant mutants can be screened for ability to confer glyphosate resistance activity to identify mutants that retain activity. Following mutagenesis, the encoded protein can be expressed recombinantly, and the activity of the protein can be determined using standard assay techniques.

Using methods such as PCR, hybridization, and the like corresponding glyphosate resistance sequences can be identified, such sequences having substantial identity to the sequences of the invention. See, for example, Sambrook J., and Russell, D. W. (2001) Molecular Cloning: A Laboratory Manual. (Cold Spring Harbor Laboratory Press, Cold Spring Harbor, N.Y.) and Innis, et al. (1990) PCR Protocols: A Guide to Methods and Applications (Academic Press, NY.).

In a hybridization method, all or part of the glyphosate resistance nucleotide sequence can be used to screen cDNA or genomic libraries. Methods for construction of such cDNA and genomic libraries are generally known in the art and are disclosed in Sambrook and Russell, 2001. The so-called hybridization probes may be genomic DNA fragments, cDNA fragments, RNA fragments, or other oligonucleotides, and may be labeled with a detectable group such as ³²P, or any other detectable marker, such as other radioisotopes, a fluorescent compound, an enzyme, or an enzyme co-factor. Probes for hybridization can be made by labeling synthetic oligonucleotides based on the known glyphosate resistance-encoding nucleotide sequence disclosed herein. Degenerate primers designed on the basis of conserved nucleotides or amino acid residues in the nucleotide sequence or encoded amino acid sequence can additionally be used. The probe typically comprises a region of nucleotide sequence that hybridizes under stringent conditions to at least about 12, preferably about 25, more preferably at least about 50, 75, 100, 125, 150, 175, 200, 250, 300, 350, or 400 consecutive nucleotides of glyphosate resistance-encoding nucleotide sequence of the invention or a fragment or variant thereof. Preparation of probes for hybridization is generally known in the art and is disclosed in Sambrook and Russell, 2001 and Sambrook et al. (1989) Molecular Cloning: A Laboratory Manual (2d ed., Cold Spring Harbor Laboratory Press, Plainview, N.Y.), both of which are herein incorporated by reference.

For example, an entire glyphosate resistance sequence disclosed herein, or one or more portions thereof, may be used as a probe capable of specifically hybridizing to corresponding glyphosate resistance sequences and messenger RNAs. To achieve specific hybridization under a variety of conditions, such probes include sequences that are unique and are preferably at least about 10 nucleotides in length, and most preferably at least about 20 nucleotides in length. Such probes may be used to amplify corresponding glyphosate resistance sequences from a chosen organism by PCR. This technique may be used to isolate additional coding sequences from a desired organism or as a diagnostic assay to determine the presence of coding sequences in an organism. Hybridization techniques include hybridization screening of plated DNA libraries (either plaques or colonies; see, for example, Sambrook et al. (1989) Molecular Cloning: A Laboratory Manual (2d ed., Cold Spring Harbor Laboratory Press, Plainview, N.Y.).

Hybridization of such sequences may be carried out under stringent conditions. By “stringent conditions” or “stringent hybridization conditions” is intended conditions under which a probe will hybridize to its target sequence to a detectably greater degree than to other sequences (e.g., at least 2-fold over background). Stringent conditions are sequence-dependent and will be different in different circumstances. By controlling the stringency of the hybridization and/or washing conditions, target sequences that are 100% complementary to the probe can be identified (homologous probing). Alternatively, stringency conditions can be adjusted to allow some mismatching in sequences so that lower degrees of similarity are detected (heterologous probing). Generally, a probe is less than about 1000 nucleotides in length, preferably less than 500 nucleotides in length.

Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. Exemplary low stringency conditions include hybridization with a buffer solution of 30 to 35% formamide, 1 M NaCl, 1% SDS (sodium dodecyl sulphate) at 37° C., and a wash in 1× to 2×SSC (20×SSC=3.0 M NaCl/0.3 M trisodium citrate) at 50 to 55° C. Exemplary moderate stringency conditions include hybridization in 40 to 45% formamide, 1.0 M NaCl, 1% SDS at 37° C., and a wash in 0.5× to 1×SSC at 55 to 60° C. Exemplary high stringency conditions include hybridization in 50% formamide, 1 M NaCl, 1% SDS at 37° C., and a wash in 0.1×SSC at 60 to 65° C. Optionally, wash buffers may comprise about 0.1% to about 1% SDS. Duration of hybridization is generally less than about 24 hours, usually about 4 to about 12 hours.

Specificity is typically the function of post-hybridization washes, the critical factors being the ionic strength and temperature of the final wash solution. For DNA-DNA hybrids, the Tₘ can be approximated from the equation of Meinkoth and Wahl (1984) Anal. Biochem. 138:267-284: Tₘ=81.5° C.+16.6 (log M)+0.41 (% GC)−0.61 (% form)−500/L; where M is the molarity of monovalent cations, % GC is the percentage of guanosine and cytosine nucleotides in the DNA, % form is the percentage of formamide in the hybridization solution, and L is the length of the hybrid in base pairs. The Tₘ is the temperature (under defined ionic strength and pH) at which 50% of a complementary target sequence hybridizes to a perfectly matched probe. Tₘ is reduced by about 1° C. for each 1% of mismatching; thus, Tₘ, hybridization, and/or wash conditions can be adjusted to hybridize to sequences of the desired identity. For example, if sequences with ≧90% identity are sought, the Tₘ can be decreased 10° C. Generally, stringent conditions are selected to be about 5° C. lower than the thermal melting point (Tₘ) for the specific sequence and its complement at a defined ionic strength and pH. However, severely stringent conditions can utilize a hybridization and/or wash at 1, 2, 3, or 4° C. lower than the thermal melting point (Tₘ); moderately stringent conditions can utilize a hybridization and/or wash at 6, 7, 8, 9, or 10° C. lower than the thermal melting point (Tₘ); low stringency conditions can utilize a hybridization and/or wash at 11, 12, 13, 14, 15, or 20° C. lower than the thermal melting point (Tₘ). Using the equation, hybridization and wash compositions, and desired Tₘ, those of ordinary skill will understand that variations in the stringency of hybridization and/or wash solutions are inherently described. If the desired degree of mismatching results in a Tₘ of less than 45° C. (aqueous solution) or 32° C. (formamide solution), it is preferred to increase the SSC concentration so that a higher temperature can be used. An extensive guide to the hybridization of nucleic acids is found in Tijssen (1993) Laboratory Techniques in Biochemistry and Molecular Biology—Hybridization with Nucleic Acid Probes, Part I, Chapter 2 (Elsevier, N.Y.); and Ausubel et al., eds. (1995) Current Protocols in Molecular Biology, Chapter 2 (Greene Publishing and Wiley-Interscience, New York). See Sambrook et al. (1989) Molecular Cloning: A Laboratory Manual (2d ed., Cold Spring Harbor Laboratory Press, Plainview, N.Y.).

### Isolated Proteins and Variants and Fragments Thereof

Glyphosate resistance proteins are also encompassed within the present invention. By “glyphosate resistance protein” is intended a protein having the amino acid sequence set forth in SEQ ID NO:3, 19, or 21. Fragments, biologically active portions, and variants thereof are also provided, and may be used to practice the methods of the present invention.

“Fragments” or “biologically active portions” include polypeptide fragments comprising a portion of an amino acid sequence encoding a glyphosate resistance protein as set forth in SEQ ID NO:3, 19, or 21, and that retains glyphosate resistance activity. A biologically active portion of a glyphosate resistance protein can be a polypeptide that is, for example, 10, 25, 50, 100 or more amino acids in length. Such biologically active portions can be prepared by recombinant techniques and evaluated for glyphosate resistance activity. Methods for measuring glyphosate resistance activity are well known in the art. See, for example, U.S. Pat. Nos. 4,535,060, and 5,188,642, each of which are herein incorporated by reference in their entirety. As used here, a fragment comprises at least 8 contiguous amino acids of SEQ ID NO:3, 19, or 21. The invention encompasses other fragments, however, such as any fragment in the protein greater than about 10, 20, 30, 50, 100, 150, 200, 250, 300, 350, 400, 450, 500, 550, or 600 amino acids.

By “variants” is intended proteins or polypeptides having an amino acid sequence that is at least about 60%, 65%, preferably about 70%, 75%, more preferably, 80%, 85%, most preferably 90%, 91%, 92%, 93%, 94%, 95%, 96%, 97%, 98% or 99% identical to the amino acid sequence of SEQ ID NO:3, 6, 8, 11, 19, or 21. Variants also include polypeptides encoded by a nucleic acid molecule that hybridizes to the nucleic acid molecule of SEQ ID NO:1, 2, 4, 5, 7, 9, 10, 18, or 21, or a complement thereof, under stringent conditions. Variants include polypeptides that differ in amino acid sequence due to mutagenesis. Variant proteins encompassed by the present invention are biologically active, that is they continue to possess the desired biological activity of the native protein, that is, retaining glyphosate resistance activity. Methods for measuring glyphosate resistance activity are well known in the art. See, for example, U.S. Pat. Nos. 4,535,060, and 5,188,642, each of which are herein incorporated by reference in their entirety.

### Altered or Improved Variants

It is recognized that DNA sequences of GDC-1 may be altered by various methods, and that these alterations may result in DNA sequences encoding proteins with amino acid sequences different than that encoded by GDC-1. This protein may be altered in various ways including amino acid substitutions, deletions, truncations, and insertions. Methods for such manipulations are generally known in the art. For example, amino acid sequence variants of the GDC-1 protein can be prepared by mutations in the DNA. This may also be accomplished by one of several forms of mutagenesis and/or in directed evolution. In some aspects, the changes encoded in the amino acid sequence will not substantially affect the function of the protein. Such variants will possess the desired glyphosate resistance activity. However, it is understood that the ability of GDC-1 to confer glyphosate resistance may be improved by the use of such techniques upon the compositions of this invention. For example, one may express GDC-1 in host cells that exhibit high rates of base misincorporation during DNA replication, such as XL-1 Red (Stratagene). After propagation in such strains, one can isolate the GDC-1 DNA (for example by preparing plasmid DNA, or by amplifying by PCR and cloning the resulting PCR fragment into a vector), culture the GDC-1 mutations in a non-mutagenic strain, and identify mutated GDC-1 genes with improved resistance to glyphosate, for example by growing cells in increasing concentrations of glyphosate and testing for clones that confer ability to tolerate increased concentrations of glyphosate.

Alternatively, alterations may be made to the protein sequence of many proteins at the amino or carboxy terminus without substantially affecting activity. This can include insertions, deletions, or alterations introduced by modern molecular methods, such as PCR, including PCR amplifications that alter or extend the protein coding sequence by virtue of inclusion of amino acid encoding sequences in the oligonucleotides utilized in the PCR amplification. Alternatively, the protein sequences added can include entire protein-coding sequences, such as those used commonly in the art to generate protein fusions. Such fusion proteins are often used to (1) increase expression of a protein of interest (2) introduce a binding domain, enzymatic activity, or epitope to facilitate either protein purification, protein detection, or other experimental uses known in the art (3) target secretion or translation of a protein to a subcellular organelle, such as the periplasmic space of Gram-negative bacteria, or the endoplasmic reticulum of eukaryotic cells, the latter of which often results in glycosylation of the protein.

Variant nucleotide and amino acid sequences of the present invention also encompass sequences derived from mutagenic and recombinogenic procedures such as DNA shuffling. With such a procedure, one or more different glyphosate resistance protein coding regions can be used to create a new glyphosate resistance protein possessing the desired properties. In this manner, libraries of recombinant polynucleotides are generated from a population of related sequence polynucleotides comprising sequence regions that have substantial sequence identity and can be homologously recombined in vitro or in vivo. For example, using this approach, sequence motifs encoding a domain of interest may be shuffled between the glyphosate resistance gene of the invention and other known glyphosate resistance genes to obtain a new gene coding for a protein with an improved property of interest, such as an increased glyphosate resistance activity. Strategies for such DNA shuffling are known in the art. See, for example, Stemmer (1994) Proc. Natl. Acad. Sci. USA 91:10747-10751; Stemmer (1994) Nature 370:389-391; Crameri et al. (1997) Nature Biotech. 15:436-438; Moore et al. (1997) J. Mol. Biol. 272:336-347; Zhang et al. (1997) Proc. Natl. Acad. Sci. USA 94:4504-4509; Crameri et al. (1998) Nature 391:288-291; and U.S. Pat. Nos. 5,605,793 and 5,837,458.

### Transformation of Bacterial or Plant Cells

In one aspect of the invention, the GDC-1 gene is useful as a marker to assess transformation of bacterial or plant cells. Transformation of bacterial cells is accomplished by one of several techniques known in the art, not limited to electroporation, or chemical transformation (See for example Ausubel (ed.), Current Protocols in Molecular Biology, John Wiley and Sons, Inc. (1994)). Markers conferring resistance to toxic substances are useful in identifying transformed cells (having taken up and expressed the test DNA) from non-transformed cells (those not containing or not expressing the test DNA). By engineering GDC-1 to be (1) expressed from a bacterial promoter known to stimulate transcription in the organism to be tested, (2) properly translated to generate an intact GDC-1 peptide, and (3) placing the cells in an otherwise toxic concentration of glyphosate, one can identify cells that have been transformed with DNA by virtue of their resistance to glyphosate.

Transformation of plant cells can be accomplished in similar fashion. First, one engineers the GDC-1 gene in a way that allows its expression in plant cells. The glyphosate resistance sequences of the invention may be provided in expression cassettes for expression in the plant of interest. The cassette will include 5′ and 3′ regulatory sequences operably linked to a sequence of the invention. By “operably linked” is intended a functional linkage between a promoter and a second sequence, wherein the promoter sequence initiates and mediates transcription of the DNA sequence corresponding to the second sequence. Generally, operably linked means that the nucleic acid sequences being linked are contiguous and, where necessary to join two protein coding regions, contiguous and in the same reading frame. The cassette may additionally contain at least one additional gene to be cotransformed into the organism. Alternatively, the additional gene(s) can be provided on multiple expression cassettes. The organization of such constructs is well known in the art.

Such an expression cassette is provided with a plurality of restriction sites for insertion of the glyphosate resistance sequence to be under the transcriptional regulation of the regulatory regions. The expression cassette will include in the 5′-3′ direction of transcription, a transcriptional and translational initiation region (i.e., a promoter), a DNA sequence of the invention, and a transcriptional and translational termination region (i.e., termination region) functional in plants. The promoter may be native or analogous, or foreign or heterologous, to the plant host and/or to the DNA sequence of the invention. Additionally, the promoter may be the natural sequence or alternatively a synthetic sequence. Where the promoter is “native” or “homologous” to the plant host, it is intended that the promoter is found in the native plant into which the promoter is introduced. Where the promoter is “foreign” or “heterologous” to the DNA sequence of the invention, it is intended that the promoter is not the native or naturally occurring promoter for the operably linked DNA sequence of the invention.

The termination region may be native with the transcriptional initiation region, may be native with the operably-linked DNA sequence of interest, may be native with the plant host, or may be derived from another source (i.e., foreign or heterologous to the promoter, the DNA sequence of interest, the plant host, or any combination thereof). Convenient termination regions are available from the Ti-plasmid of A. tumefaciens, such as the octopine synthase and nopaline synthase termination regions. See also Guerineau et al. (1991) Mol. Gen. Genet. 262:141-144; Proudfoot (1991) Cell 64:671-674; Sanfacon et al. (1991) Genes Dev. 5:141-149; Mogen et al. (1990) Plant Cell 2:1261-1272; Munroe et al. (1990) Gene 91:151-158; Ballas et al. (1989) Nucleic Acids Res. 17:7891-7903; and Joshi et al. (1987) Nucleic Acid Res. 15:9627-9639.

Where appropriate, the gene(s) may be optimized for increased expression in the transformed host cell. That is, the genes can be synthesized using host cell-preferred codons for improved expression, or may be synthesized using codons at a host-preferred codon usage frequency. Generally, the GC content of the gene will be increased. See, for example, Campbell and Gowri (1990) Plant Physiol. 92:1-11 for a discussion of host-preferred codon usage. Methods are known in the art for synthesizing host-preferred genes. See, for example, U.S. Pat. Nos. 6,320,100; 6,075,185; 5,380,831; and 5,436,391, U.S. Published Application Nos. 20040005600 and 20010003849, and Murray et al. (1989) Nucleic Acids Res. 17:477-498, herein incorporated by reference.

In some instances, it may be useful to engineer the gene such that the resulting peptide is secreted, or otherwise targeted within the plant cell. For example, the gene can be engineered to contain a signal peptide to facilitate transfer of the peptide to the endoplasmic reticulum. It may also be preferable to engineer the plant expression cassette to contain an intron, such that mRNA processing of the intron is required for expression. In one embodiment, the nucleic acids of interest are targeted to the chloroplast for expression. In this manner, where the nucleic acid of interest is not directly inserted into the chloroplast, the expression cassette will additionally contain a nucleic acid encoding a transit peptide to direct the gene product of interest to the chloroplasts. Such transit peptides are known in the art. See, for example, Von Heijne et al. (1991) Plant Mol. Biol. Rep. 9:104-126; Clark et al. (1989) J. Biol. Chem. 264:17544-17550; Della-Cioppa et al. (1987) Plant Physiol. 84:965-968; Romer et al. (1993) Biochem. Biophys. Res. Commun. 196:1414-1421; and Shah et al. (1986) Science 233:478-481.

Methods for transformation of chloroplasts are known in the art. See, for example, Svab et al. (1990) Proc. Natl. Acad. Sci. USA 87:8526-8530; Svab and Maliga (1993) Proc. Natl. Acad. Sci. USA 90:913-917; Svab and Maliga (1993) EMBO J. 12:601-606. The method relies on particle gun delivery of DNA containing a selectable marker and targeting of the DNA to the plastid genome through homologous recombination. Additionally, plastid transformation can be accomplished by transactivation of a silent plastid-bome transgene by tissue-preferred expression of a nuclear-encoded and plastid-directed RNA polymerase. Such a system has been reported in McBride et al. (1994) Proc. Natl. Acad. Sci. USA 91:7301-7305.

The nucleic acids of interest to be targeted to the chloroplast may be optimized for expression in the chloroplast to account for differences in codon usage between the plant nucleus and this organelle. In this manner, the nucleic acids of interest may be synthesized using chloroplast-preferred codons. See, for example, U.S. Pat. No. 5,380,831, herein incorporated by reference.

Typically this ‘plant expression cassette’ will be inserted into a ‘plant transformation vector’. This plant transformation vector may be comprised of one or more DNA vectors needed for achieving plant transformation. For example, it is a common practice in the art to utilize plant transformation vectors that are comprised of more than one contiguous DNA segment. These vectors are often referred to in the art as ‘binary vectors’. Binary vectors as well as vectors with helper plasmids are most often used for Agrobacterium-mediated transformation, where the size and complexity of DNA segments needed to achieve efficient transformation is quite large, and it is advantageous to separate functions onto separate DNA molecules. Binary vectors typically contain a plasmid vector that contains the cis-acting sequences required for T-DNA transfer (such as left border and right border), a selectable marker that is engineered to be capable of expression in a plant cell, and a ‘gene of interest’ (a gene engineered to be capable of expression in a plant cell for which generation of transgenic plants is desired). Also present on this plasmid vector are sequences required for bacterial replication. The cis-acting sequences are arranged in a fashion to allow efficient transfer into plant cells and expression therein. For example, the selectable marker gene and the gene of interest are located between the left and right borders. Often a second plasmid vector contains the trans-acting factors that mediate T-DNA transfer from Agrobacterium to plant cells. This plasmid often contains the virulence functions (Vir genes) that allow infection of plant cells by Agrobacterium, and transfer of DNA by cleavage at border sequences and vir-mediated DNA transfer, as in understood in the art (Hellens and Mullineaux (2000) Trends in Plant Science 5:446-451). Several types of Agrobacterium strains (e.g. LBA4404, GV3101, EHA101, EHA105, etc.) can be used for plant transformation. The second plasmid vector is not necessary for transforming the plants by other methods such as microprojection, microinjection, electroporation, polyethelene glycol, etc. Many types of vectors can be used to transform plant cells for achieving glyphosate resistance.

In general, plant transformation methods involve transferring heterologous DNA into target plant cells (e.g. immature or mature embryos, suspension cultures, undifferentiated callus, protoplasts, etc.), followed by applying a maximum threshold level of appropropriate selection (depending on the selectable marker gene and in this case “glyphosate”) to recover the transformed plant cells from a group of untransformed cell mass. Explants are typically transferred to a fresh supply of the same medium and cultured routinely. Subsequently, the transformed cells are differentiated into shoots after placing on regeneration medium supplemented with a maximum threshold level of selecting agent (e.g. “glyphosate”). The shoots are then transferred to a selective rooting medium for recovering rooted shoot or plantlet. The transgenic plantlet then grow into mature plant and produce fertile seeds (e.g. Hiei et al. (1994) The Plant Journal 6:271-282; Ishida et al. (1996) Nature Biotechnology 14:745-750). Explants are typically transferred to a fresh supply of the same medium and cultured routinely. A general description of the techniques and methods for generating transgenic plants are found in Ayres and Park (1994) Critical Reviews in Plant Science 13:219-239 and Bommineni and Jauhar (1997) Maydica 42:107-120. Since the transformed material contains many cells; both transformed and non-transformed cells are present in any piece of subjected target callus or tissue or group of cells. The ability to kill non-transformed cells and allow transformed cells to proliferate results in transformed plant cultures. Often, the ability to remove non-transformed cells is a limitation to rapid recovery of transformed plant cells and successful generation of transgenic plants.

Generation of transgenic plants may be performed by one of several methods, including but not limited to introduction of heterologous DNA by Agrobacterium into plant cells (Agrobacterium-mediated transformation). Bombardment of plant cells with heterologous foreign DNA adhered to particles including aerosol beam transformation (U.S. Published Application No. 20010026941; U.S. Pat. No. 4,945,050; International Publication No. WO 91/00915; U.S. Published Application No. 2002015066), and various other non-particle direct-mediated methods (e.g. Hiei et al. (1994) The Plant Journal 6:271-282; Ishida et al. (1996) Nature Biotechnology 14:745-750; Ayres and Park (1994) Critical Reviews in Plant Science 13:219-239; Bommineni and Jauhar (1997) Maydica 42:107-120) to transfer DNA.

Following integration of heterologous foreign DNA into plant cells, one then applies a maximum threshold level of glyphosate in the medium to kill the untransformed cells and separate and proliferate the putatively transformed cells that survive from this selection treatment by transferring regularly to a fresh medium. By continuous passage and challenge with glyphosate, one identifies and proliferates the cells that are transformed with the plasmid vector. Then molecular and biochemical methods will be used for confirming the presence of the integrated heterologous gene of interest in the genome of transgenic plant.

The cells that have been transformed may be grown into plants in accordance with conventional ways. See, for example, McCormick et al. (1986) Plant Cell Reports 5:81-84. These plants may then be grown, and either pollinated with the same transformed strain or different strains, and the resulting hybrid having constitutive expression of the desired phenotypic characteristic identified. Two or more generations may be grown to ensure that expression of the desired phenotypic characteristic is stably maintained and inherited and then seeds harvested to ensure expression of the desired phenotypic characteristic has been achieved. In this manner, the present invention provides transformed seed (also referred to as “transgenic seed”) having a nucleotide construct of the invention, for example, an expression cassette of the invention, stably incorporated into their genome.

### Evaluation of Plant Transformation

Following introduction of heterologous foreign DNA into plant cells, the transformation or integration of heterologous gene in the plant genome is confirmed by various methods such as analysis of nucleic acids, proteins and metabolites associated with the integrated gene.

PCR Analysis: PCR analysis is a rapid method to screen transformed cells, tissue or shoots for the presence of incorporated gene at the earlier stage before transplanting into the soil (Sambrook and Russell, 2001). PCR is carried out using oligonucleotide primers specific to the gene of interest or Agrobacterium vector background, etc.

Southern Analysis: Plant transformation is confirmed by Southern blot analysis of genomic DNA (Sambrook and Russell, 2001). In general, total DNA is extracted from the transformant, digested with appropriate restriction enzymes, fractionated in an agarose gel and transferred to a nitrocellulose or nylon membrane. The membrane or “blot” then is probed with, for example, radiolabeled ³²P target DNA fragment to confirm the integration of introduced gene in the plant genome according to standard techniques (Sambrook and Russell, 2001. Molecular Cloning: A Laboratory Manual. Cold Spring Harbor Laboratory Press, Cold Spring Harbor, N.Y.

Northern Analysis: RNA is isolated from specific tissues of transformant, fractionated in a formaldehyde agarose gel, blotted onto a nylon filter according to standard procedures that are routinely used in the art (Sambrook, J., and Russell, D. W. 2001. Molecular Cloning: A Laboratory Manual, Cold Spring Harbor Laboratory Press, Cold Spring Harbor, N.Y.). Expression of RNA encoded by GDC-1 is then tested by hybridizing the filter to a radioactive probe derived from a GDC, by methods known in the art (Sambrook and Russell, 2001)

Western blot and Biochemical assays: Western blot and biochemical assays and the like may be carried out on the transgenic plants to determine the presence of protein encoded by the glyphosate resistance gene by standard procedures (Sambrook, J., and Russell, D. W. 2001. Molecular Cloning: A Laboratory Manual, Cold Spring Harbor Laboratory Press, Cold Spring Harbor, N.Y.) using antibodies that bind to one or more epitopes present on the glyphosate resistance protein.

### Transgenic Plants

In another aspect of the invention, one may generate transgenic plants expressing GDC-1 that are more resistant to high concentrations of glyphosate than non-transformed plants. Methods described above by way of example may be utilized to generate transgenic plants, but the manner in which the transgenic plant cells are generated is not critical to this invention. Methods known or described in the art such as Agrobacterium-mediated transformation, biolistic transformation, and non-particle-mediated methods may be used at the discretion of the experimenter. Plants expressing GDC-1 may be isolated by common methods described in the art, for example by transformation of callus, selection of transformed callus, and regeneration of fertile plants from such transgenic callus. In such process, GDC-1 may be used as selectable marker. Alternatively, one may use any gene as a selectable marker so long as its expression in plant cells confers ability to identify or select for transformed cells. Genes known to function effectively as selectable markers in plant transformation are well known in the art.

The following examples are offered by way of illustration and not by way of limitation.

### EXPERIMENTAL

### Example 1

### Isolation of ATX6394

Glyphosate-resistant fungi were isolated by plating samples of soil on Enriched Minimal Media (EMM) containing glyphosate as the sole source of phosphorus. Since EMM contains no aromatic amino acids, a strain must be resistant to glyphosate in order to grow on this media.

Two grams of soil was suspended in approximately 30 ml of water, and sonicated for 30 seconds in an Aquasonic sonicator water bath. The sample was vortexed for 5 seconds and permitted to settle for 60 seconds. This process was repeated 3 times. 100 μl of this suspension was added to 2 ml of Enriched Minimal Media II (EMM II) supplemented with 4 mM glyphosate (pH 6.0) EMMII contains Solution A (In 900 mls: 10 g sucrose (or other carbon source), 2 g NaNO₃, 1.0 ml 0.8 M MgSO₄, 1.0 ml 0.1 M CaCl₂, 1.0 ml Trace Elements Solution (In 100 ml of 1000× solution: 0.1 g FeSO₄.7H₂O, 0.5 mg CuSO₄.5H₂O, 1.0 mg H₃BO₃, 1.0 mg MnSO₄.5H₂O, 7.0 mg ZnSO₄.7H₂O, 1.0 mg MoO₃,4.0 g KCl)) and Solution B (In 100 mls: 0.21 g Na₂HPO₄, 0.09 g NaH₂PO₄, pH 7.0). The culture was shaken on a tissue culture roller drum for eight days at 21° C. and then transferred into 2 ml of fresh EMMII containing 4 mM glyphosate as the only phosphorus source. After five days, the culture was plated onto solid media by streaking a 1 μl loop onto the surface of agar plate containing EMMII agar containing 5 mM glyphosate as the sole phosphorus source. The plate was sealed with parafilm and incubated until suitable growth was attained. Fresh plates were inoculated by agar plugs to isolate the fungus into pure culture.

One particular strain, designated ATX6394, was selected due to its ability to grow in the presence of high glyphosate concentrations.

### Example 2

### Construction of cDNA Library from Strain ATX6394

ATX6394 was grown in (liquid media L+phosphorous) containing 5 mM glyphosate, and total RNA was isolated using Trizol reagent (Invitrogen). poly(A)+ mRNA was isolated from total RNA using Poly(A) Purist mRNA Purification kit (Ambion). cDNA was synthesized from polyA+ mRNA using ZAP cDNA Synthesis kit from Stratagene, and cloned into the lambda Zap II expression vector (Stratagene).

### Example 3

### In vivo Excision of cDNA Clones

The ATX6394 cDNA library was excised in bulk as per manufacturers protocol (Stratagene), transfected into the SOLR strain of E. coli (Stratagene), plated directly onto M9 minimal media plates containing thiamine, proline, ampicillin and 5 mM glyphosate and incubated at 37° C. (M9 media contains 30 g Na₂HPO₄, 15 g KH₂PO₄, 5 g NH₄Cl, 2.5 g NaCl, and 15 mg CaCl₂).

### Example 4

### Identification of cDNA Clones Conferring Glyphosate Resistance in E. coli

Following 2 days growth, 51 colonies had grown in the presence of 5 mM glyphosate, and these clones were selected for further study. Plasmid DNA from 48 of the 51 positive clones was isolated and transformed into the alternate host strain XL-1 Blue MRF′ (Stratagene) and plasmid DNA was prepared for sequencing.

We determined the DNA sequence of 48 clones conferring glyphosate resistance (5 mM). Three clones (#23, 35, 59) were found to represent the same open reading frame. Therefore we designated this open reading frame GDC-1. The nucleotide sequences of clones #23, 35, and 59 are provided in SEQ ID NOS:4, 7, and 9 respectively.

### Example 5

### Isolation of Full-length GDC-1 Construct (GDC-1 (Full))

Comparison of GDC-1 (29) GDC-1(35) and GDC-1 (59) suggested that these clones did not represent the entire cDNA for the GDC-1 mRNA. To generate a full length GDC-1 clone, we performed 5′ RACE using the SMART RACE cDNA Amplification kit (BD Biosciences) to amplify the 5′ end of the GDC-1 from ATX6394 poly(A)+ mRNA. Oligo [SMARTgrg3.rev 5′TCCCAGATGCCAAAGTTGGCTGTTCCAGTC 3′]; SEQ ID NO:12 was derived from the sequence of GDC-1 (#35). We cut the resultant PCR product with HindIII and ligated this to the existing GDC-1(59) cDNA in pBluescript to generate the full length cDNA, referred to herein as GDC-1(full). The DNA sequence of GDC-1 (full) was determined, and found to contain a complete protein-coding region. This coding region is referred to herein as GDC-1. Amino acid sequences resulting from the translation of the GDC-1 gene are provided in SEQ ID NOS:3, 19, and 21.

GDC-1(59) consists of amino acid residues 118 to 575 of GDC-1(full) (SEQ ID NO:19). GDC-1(35) consists of amino acid residues 331 to 556 of GDC-1(full) (SEQ ID NO:19). GDC-1(23) consists of amino acid residues 379 to 575 of GDC-1(full) (SEQ ID NO:19).

### Example 6

### Growth of GDC-1 Clones in Liquid Cultures Containing Glyphosate

Starter cultures of E. coli containing GDC-1(35), GDC-1(Fl) or vector alone were grown 6 hours in LB media, then diluted 1:100 into 1 ml M9 minimal media containing 0, 1, 2, 5, 10, 20 and 30 mM glyphosate and grown overnight at 37° C. At 16 h, OD600 was measured for each (in triplicate).

TABLE 1
Growth of GDC-1 containing strains in high
concentrations of glyphosate

| | OD₆₀₀ after 16 hours | | | | | |
|---|---|---|---|---|---|---|
| [Glyphosate] | Vector | SD | GDC-1(35) | SD | GDC-1(Full) | SD |
| 0 | 0.077 | 0.003 | 0.123 | 0.003 | 0.099 | 0.002 |
| 20 mM | 0.043 | 0.001 | 0.094 | 0.003 | 0.098 | 0.005 |
| 30 mM | 0.039 | 0.002 | 0.067 | 0.005 | 0.102 | 0.001 |

### Example 7

### Disruption of GDC-1 ORF Eliminates Glyphosate Resistance

To confirm that GDC-1 ORF is responsible for conferring glyphosate resistance, we engineered a mutant of GDC-1(35), and tested its ability to confer glyphosate resistance. The GDC-1(35) construct contains a single recognition site for HindIII restriction enzyme. GDC-1(35) was digested with the restriction enzyme Hind III, and the resulting recessed 3′ ends extended by incubating with T4 DNA polymerase and dNTPs, as known in the art (Sambrook). The resulting molecules were then religated using T4 DNA ligase (Maniatis). The religated molecules were identified by min-prep of transformed clones, and the DNA was sequenced. The resulting clone, GDC-1(35-H3mut), contains a four nucleotide insertion in the GDC-1 open reading frame. This four nucleotide insertion leads to the premature termination of translation of the GDC-1(35) protein at a premature stop codon at nucleotides 1451-1453 of GDC-1 full length sequence.

TABLE 2
Glyphosate resistance of GDC-1(35) and
the mutant GDC-1 (35-H3mut)

| | M9 media + Amp + |
|---|---|
| | 10 mM Glyphosate |
| Vector (pBluescript SK+) | − |
| GDC-1(35) | +++ |
| GDC-1(35-H3mut) | − |

### Example 8

### GDC-1 is a TPP-binding Decarboxylase

The predicted amino acid sequence of GDC-1 was compared to the non-redundant database of sequences maintained by the National Center for Biotechnology Information (NCBI), using the BLAST2 algorithm (Altschul et al. (1990) J. Mol. Biol. 215:403-410; Altschul et al. (1997) Nucleic Acids Res. 25:3389-3402; Gish and States (1993) Nature Genet. 3:266-272). Comparison of GDC-1 with public DNA and amino acid databases, such as the non-redundant database of GenBank, the Swissprot database, and the ‘pat’ database of GenBank show that GDC-1 encodes a novel protein. Results from a BLAST search of the NCBI nr database are shown in Table 3. The sequences obtained using the Genbank Accession Nos. provided are herein incorporated by reference in their entirety. The results of BLAST searches identified homology between the predicted GDC-1 open reading frame (SEQ ID NO:3) and several known proteins. The highest scoring amino acid sequences from this search were aligned with GDC-1 using ClustalW algorithm (Higgins et al. (1994) Nucleic Acids Res. 22:4673-4680) [as incorporated into the program ALIGNX module of the vector NTi Program Suite, Informax, Inc.]. After alignment with ClustalW, the percent amino acid identity was assessed. The protein encoded by GDC-1 has homology to several members of the fungal pyruvate decarboxylase enzyme family. The highest protein homology identified is the Aspergillus oryzae pyruvate decarboxylase (pdcA) gene. GDC-1 also shares homology with indole-3 pyruvate decarboxylases, found in bacteria such as Salmonella typhimurium. A similar search of the patent database at NCBI also identifies proteins with homology to GDC-1, though proteins identified in this search are less related to GDC-1. The percent amino acid identity of GDC-1 with members of these protein classes is shown in Table 4.

Further analysis of GDC-1 sequence shows that GDC-1 contains conserved domains characteristic of proteins that utilize Thiamine Pyrophosphate (TPP) as a cofactor. These domains are collectively and singly referred to as a “TPP binding domain”. Analysis of GDC-1 sequence shows that amino acids 13-187 of SEQ ID NOS:3, 19, and 21 constitute an N-terminal domain of TPP-binding domain, amino acids 375-547 of SEQ ID NOS:3, 19, and 21 constitute a central domain of TPP-binding domain, and amino acids 209-348 of SEQ ID NOS:3, 19, and 21 constitute a C-terminal domain of TPP-binding domain. It is understood that these amino acid coordinates are only approximations of the location of such domains as judged by homology with known TPP binding proteins, and are not limiting to the invention. An alignment of GDC-1 with other known TPP-binding proteins is shown in FIG. 3.

TABLE 3
High scoring open reading frames from BLAST
search of NCBI nr database

| Genbank Accession No. | Organism | Gene Description |
|---|---|---|
| gi\|4323052\|gb\|AF098293.1\|AF098293 | Aspergillus oryzae | pyruvate decarboxylase |
| | | (pdcA) |
| gi\|2160687\|gb\|U73194.1\|ENU73194 | Emericella nidulans | pyruvate decarboxylase |
| | | (pdcA) |
| gi\|25992751\|gb\|AF545432.1\| | Candida glabrata | pyruvate decarboxylase |
| | | (PDC) |
| gi\|4115\|emb\|X55905.1\|SCPDC6 | Saccharomyces | PDC6 gene for pyruvate |
| | cerivisiae | decarboxylase |
| gi\|173308\|gb\|L09727.1\|YSKPDC1A | Kluyveromyces | pyruvate decarboxylase |
| | marxianus | (PDC1) |
| gi\|535343\|gb\|U13635.1\|HUU13635 | Hanseniaspora | pyruvate decarboxylase |
| | uvarum | (PDC) |
| gi\|4113\|emb\|X15668.1\|SCPDC5 | Saccharomyces | PDC5 gene for pyruvate |
| | cerivisiae | decarboxylase |
| | | (EC4.1.1.1.) |
| gi\|452688\|emb\|X77316.1\|SCPDC1A | Saccharomyces | PDC1 |
| | cerivisiae | |

TABLE 4
Percent identity of GDC-1 to related proteins
from various fungi and bacteria

| | | % amino |
|---|---|---|
| | | acid |
| Organism | Gene Product | identity |
| Aspergillus oryzae | Pyruvate decarboxylase | 58% |
| Emericalla nidulans | Pyruvate decarboxylase | 56% |
| Candida glabrata | Pyruvate decarboxylase | 49% |
| Kluyveromyces marxianus | Pyruvate decarboxylase | 47% |
| Saccharomyces cerevisiae | Pyruvate decarboxylase PDC1 | 46% |
| Saccharomyces cerevisiae | Pyruvate decarboxylase PDC5 | 47% |
| Saccharomyces cerevisiae | Pyruvate decarboxylase PDC6 | 47% |
| Pichia Stipitis | Pyruvate decarboxylase PDC2 | 45% |
| Salmonella typhimurium | Indole-3 pyruvate decarboxylase | 33% |
| Neurospora crassa | Pyruvate decarboxylase | 28% |
| Nicotiana tabacum | Pyruvate decarboxylase | 28% |
| Zymomonas mobilis | Pyruvate decarboxylase | 27% |

### Example 9

### Engineering GDC-1 for Expression in E.coli

An E. coli strain expressing GDC-1 was engineered into a customized expression vector (pAX481). pAX481 contains the pBR322 origin of replication, a chloramphenicol acetyl transferase gene (for selection and maintenance of the plasmid), the lacI gene, the Ptac promoter and the rrnB transcriptional terminator. The GDC-1 open reading frame was amplified by PCR using a high fidelity DNA polymerase, as known in the art. The oligonucleotides for PCR amplification of GDC-1 were designed to place the ATG start site of the gene at the proper distance from the ribosome binding site of pAX481.

The GDC-1 PCR product was cloned into the expression vector pAX481 and transformed into E. coli XL1 Blue MRF′ to yield the plasmid pAX472. GDC-1 positive clones were identified by standard methods known in the art. The sequence of pAX472 was confirmed by DNA sequence analysis as known in the art.

### Example 10

### GDC-1 Confers Resistance to High Levels of Glyphosate

E. coli strains containing GDC-1 (pAX472) expression vector or vector control (pAX481) were grown to saturation in M63 media, and diluted into a 48-well plate by adding 40 μl of cells to 1 ml cultures. Cultures contained M63 (13.6 g KH₂PO₄; 2 g (NH₄)₂SO₄; 0.5 mg FeSO₄-7H₂O; 2.4 mg MgCl₂ in 1 liter dH₂O) supplemented with proline and thiamine, 20 ug/ml chloramphenicol, 0.5% glucose, and from 0 to 200 mM glyphosate, diluted from a 1 M stock solution. 1 mM IPTG was added to each well to induce protein expression.

Cultures were grown at 37° C. with shaking at 300 rpm. At 0 hours and at 42 hours, 300 μl of culture was withdrawn and placed into a 96-well assay plate. The absorbance of the culture at 600 nm was measured in a 96-well plate using a Spectramax 190 Spectrophotometer (Molecular Devices, Inc.). The absorbance of the cultures at 0 hours was consistently below 0.04. The table below shows the absorbance at 600 nM obtained from the individual cultures after 42 hours of incubation.

TABLE 5
GDC 1 confers glyphosate resistance upon sensitive cells

| [Gly] mM | GDC1 | Vector | Media |
|---|---|---|---|
| 0 | 1.37 | 1.28 | 0.04 |
| 25 | 1.20 | 0.21 | 0.04 |
| 50 | 1.40 | 0.21 | 0.04 |
| 75 | 1.27 | 0.16 | 0.04 |
| 100 | 1.26 | 0.22 | 0.04 |
| 125 | 1.23 | 0.20 | 0.04 |
| 150 | 1.33 | 0.20 | 0.04 |
| 200 | 1.11 | 0.22 | 0.04 |

### Example 11

### GDC-1 does not Complement an aroA Mutation in E. coli

The E. coli aroA gene codes for EPSP synthase, the target enzyme for glyphosate. EPSP synthase catalyzes the sixth step in the biosynthesis of aromatic amino acids in microbes and plants. aroA mutants that lack an EPSP synthase do not grow on minimal media that lacks aromatic amino acids (Pittard and Wallace (1966) J. Bacteriol. 91:1494-508), but can grow in rich media, such as LB. However, genes encoding EPSPS activity can restore the ability to grow on glyphosate upon aroA mutant E.coli strains. Thus, a test for genetic complementation of an aroA mutant is a highly sensitive method to test if a gene is capable of functioning as an EPSPS in E.coli. Such tests for gene function by genetic complementation are known in the art.

A deletion of the aroA gene was created in E. coli XL-1 MRF′ (Stratagene) by PCR/recombination methods known in the art and outlined by Datsenko and Wanner, (2000) Proc. Natl. Acad. Sci. USA 97:6640-6645. This system is based on the Red system that allows for chromosomal disruptions of targeted sequences. A large portion (1067 nt of the 1283 nt) of the aroA coding region was disrupted by the engineered deletion. The presence of the deletion was confirmed by PCR with several sets of oligonucleotides, and by the appearance of an aroA phenotype in the strain, referred to herein as ‘ΔaroA’. ΔaroA grows on LB media (which contains all amino acids) and grows on M63 media supplemented with phenylalanine, tryptophan, and tyrosine, but does not grow on M63 minimal media (which lacks aromatic amino acids). These results indicate that ΔaroA exhibits an aroA phenotype.

The ability of an EPSPS to complement the mutant phenotype of ΔaroA was confirmed. Clone pAX482, an E.coli expression vector containing the wild-type E.coli aroA gene, was transformed into ΔaroA, and transformed cells were selected. These cells (containing a functional aroA gene residing on a plasmid) were then plated on LB media, M63, and M63 with amino acid supplements. Where the ΔaroA mutant strain grew only on LB and M63 supplemented with aromatic amino acids, ΔaroA cells containing the functional aroA gene on a plasmid grew on all three media types.

In order to determine whether or not GDC-1 could confer complementation, plasmid pAX472, the expression vector containing GDC-1, was transformed into ΔaroA and plated on the same three types of media. Cells transformed with pAX472 were able to grow on M63 media supplemented with phenylalanine, tryptophan, and tyrosine and LB media but they were not able to grow on M63 alone. Thus, GDC-1 was not capable of complementing the aroA mutation, and thus GDC-1 is not EPSP synthase.

### Example 12

### Purification of GDC-1 Expressed as a 6×His-tagged Protein in E. coli

The GDC-1 coding region (1,728 nucleotides) was amplified by PCR using ProofStart™ DNA polymerase. Oligonucleotides used to prime PCR were designed to introduce restriction enzyme recognition sites near the 5′ and 3′ ends of the resulting PCR product. The resulting PCR product was digested with BamH I and Sal I. BamH I cleaved the PCR product at the 5′ end, and Sal I cleaved the PCR product at the 3′ end. The digested product was cloned into the 6×His-tag expression vector pQE-30 (Qiagen), prepared by digestion with BamH I and Sal I. The resulting clone, pAX623, contained GDC-1 in the same translational reading frame as, and immediately C-terminal to, the 6×His tag of pQE-30. General strategies for generating such clones, and for expressing proteins containing 6×His-tag are well known in the art.

The ability of this clone to confer glyphosate resistance was confirmed by plating cells of pAX623 onto M63 media containing 5 mM glyphosate. pAX623-containing cells gave rise to colonies, where cells containing the vector alone gave no colonies.

GDC-1 protein from pAX623-containing cells was isolated by expression of GDC-1-6×His-tagged protein in E. coli, and the resulting protein purified using Ni-NTA Superflow Resin (Qiagen) as per manufacturer's instructions.

### Example 13

### Assay of GDC-1 Pyruvate Decarboxylase Activity

100 ng of GDC-1 protein was tested for activity in a standard pyruvate decarboxylase assay (Gounaris et al. (1971) J. of Biol. Chem. 246:1302-1309). This assay is a coupled reaction, wherein the first step the pyruvate decarboxylase (PDC) converts pyruvate to acetaldehyde and CO₂. The acetaldehyde produced in this reaction is a substrate for alcohol dehydrogenase, which converts acetaldehyde and β-NADH to ethanol and β-NAD. Thus, PDC activity is detected by virtue of utilization of β-NADH as decrease in absorbance at 340 nM in a spectrophotometer. GDC-1 as well as a control enzyme (pyruvate decarboxylase, Sigma) were tested in this assay. GDC-1 showed activity as a pyruvate decarboxylase, and the reaction rate correlated with the concentration of pyruvate in the assay.

### Example 14

### Assay of GDC-1 Ability to Modify Glyphosate

The ability of GDC-1 to modify glyphosate in vitro was tested by incubating GDC-1 with a mixture of radiolabeled and non-labeled glyphosate, and analyzing the reaction products by HPLC.

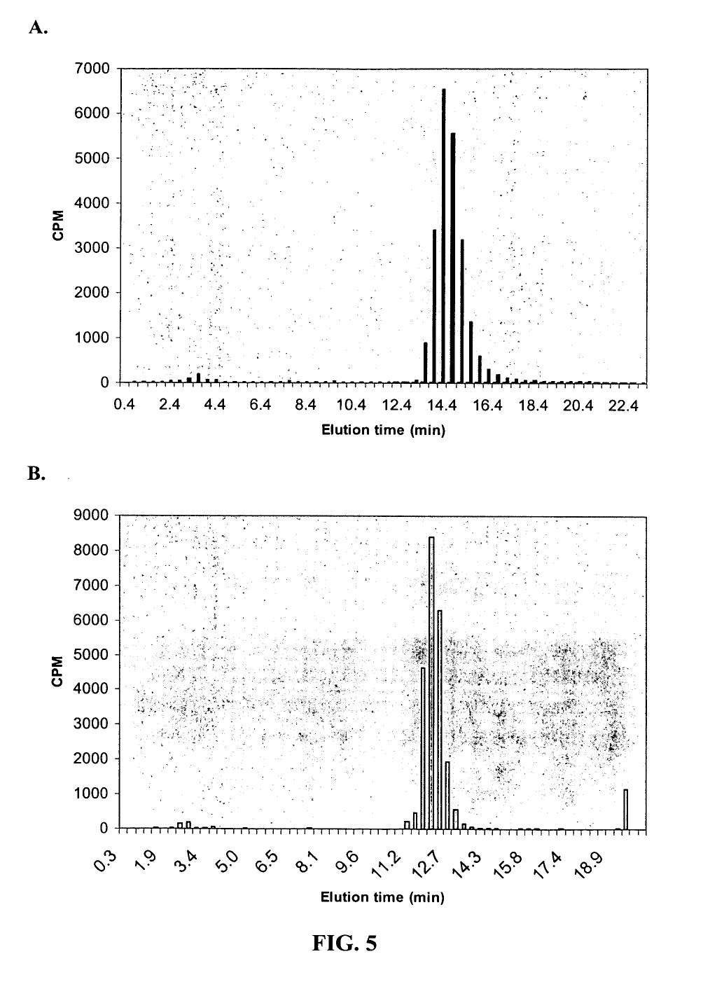

100 ng of GDC-1 purified protein was incubated with 20,000 cpm of C¹⁴-labeled glyphosate (NaOOCCH₂NH¹⁴CH₂PO₃H₂; Sigma catalog #G7014), and mixed with unlabelled glyphosate to a final concentration of 2 mM in a reaction buffer of 200 mM Na-Citrate, pH 6.0, 1 mM TPP, 2 mM MgCl₂. The reaction was allowed to proceed 60 minutes, then 5 μl was applied to an HPLC column (Dionex AminoPac PA10 analytical (and guard) column, anion exchange resin; Dionex Corporation). The column was equilibrated with 150 mM sodium hydroxide. Fractions were eluted with a sodium acetate gradient of 150-300 mM sodium acetate. Single drop (40 uL) fractions were collected, and the radioactivity present in each fraction was determined using a 96-well scintillation counter. Analysis of the resulting data shows that GDC-1 converted a portion of the labeled glyphosate to a product with an elution time of approximately 19 minutes (FIG. 5). Control experiments lacking purified GDC-1 showed no peak at this elution time.

### Example 15

### Engineering GDC-1 for Plant Transformation

The GDC-1 open reading frame (ORF) was amplified by PCR from a full-length cDNA template. HindIII restriction sites were added to each end of the ORF during PCR. Additionally, the nucleotide sequence ACC was added immediately 5′ to the start codon of the gene to increase translational efficiency (Kozak (1987) Nucleic Acids Research 15:8125-8148; Joshi (1987) Nucleic Acids Research 15:6643-6653). The PCR product was cloned and sequenced, using techniques well known in the art, to ensure that no mutations were introduced during PCR.

The plasmid containing the GDC-1 PCR product was partially digested with Hind III and the 1.7 kb Hind III fragment containing the intact ORF was isolated. (GDC-1 contains an internal Hind III site in addition to the sites added by PCR.) This fragment was cloned into the Hind III site of plasmid pAX200, a plant expression vector containing the rice actin promoter (McElroy et al. (1991) Mol. Gen. Genet. 231:150-160) and the PinII terminator (An et al. (1989) The Plant Cell 1:115-122). The promoter—gene—terminator fragment from this intermediate plasmid was subcloned into Xho I site of plasmid pSB11 (Japan Tobacco, Inc.) to form the plasmid pAX810. pAX810 is organized such that the 3.45 kb DNA fragment containing the promoter—GDC-1—terminator construct may be excised from pAX810 by double digestion with KpnI and XbaI for transformation into plants using aerosol beam injection. The structure of pAX810 was verified by restriction digests and gel electrophoresis and by sequencing across the various cloning junctions.

Plasmid pAX810 was mobilized into Agrobacterium tumifaciens strain LBA4404 which also harbored the plasmid pSB1 (Japan Tobacco, Inc.), using triparental mating procedures well known in the art, and plated on media containing spectinomycin. Plasmid pAX810 carries spectinomycin resistance but is a narrow host range plasmid and cannot replicate in Agrobacterium. Spectinomycin resistant colonies arise when pAX810 integrates into the broad host range plasmid pSB1 through homologous recombination. The cointegrate product of pSB1 and pAX810 was named pAX204 and was verified by Southern hybridization (data not shown). The Agrobacterium strain harboring pAX204 was used to transform maize by the PureIntro method (Japan Tobacco).

### Example 16

### Transformation of GDC-1 into Plant Cells

Maize ears are collected 8-12 days after pollination. Embryos are isolated from the ears, and those embryos 0.8-1.5 mm in size are used for transformation. Embryos are plated scutellum side-up on a suitable incubation media, such as DN62A5S media (3.98 g/L N6 Salts; 1 mL/L (of 1000×Stock) N6 Vitamins; 800 mg/L L-Asparagine; 100 mg/L Myo-inositol; 1.4 g/L L-Proline; 100 mg/L Casaminoacids; 50 g/L sucrose; 1 mL/L (of 1 mg/mL Stock) 2,4-D). However, media and salts other than DN62A5S are suitable and are known in the art. Embryos are incubated overnight at 25° C. in the dark.

The resulting explants are transferred to mesh squares (30-40 per plate), transferred onto osmotic media for 30-45 minutes, and then transferred to a beaming plate (see, for example, PCT Publication No. WO/0138514 and U.S. Pat. No. 5,240,842).

DNA constructs designed to express GDC-1 in plant cells are accelerated into plant tissue using an aerosol beam accelerator, using conditions essentially as described in PCT Publication No. WO/0138514. After beaming, embryos are incubated for 30 min on osmotic media, and placed onto incubation media overnight at 25° C. in the dark. To avoid unduly damaging beamed explants, they are incubated for at least 24 hours prior to transfer to recovery media. Embryos are then spread onto recovery period media, for 5 days, 25° C. in the dark, then transferred to a selection media. Explants are incubated in selection media for up to eight weeks, depending on the nature and characteristics of the particular selection utilized. After the selection period, the resulting callus is transferred to embryo maturation media, until the formation of mature somatic embryos is observed. The resulting mature somatic embryos are then placed under low light, and the process of regeneration is initiated by methods known in the art. The resulting shoots are allowed to root on rooting media, and the resulting plants are transferred to nursery pots and propagated as transgenic plants.

### Materials

### DN62A5S Media

| Components | per liter | Source |
|---|---|---|
| Chu'S N6 Basal | 3.98 g/L | Phytotechnology Labs |
| Salt Mixture | | |
| (Prod. No. C | | |
| 416) | | |
| Chu's N6 | 1 mL/L (of 1000 × | Phytotechnology Labs |
| Vitamin | Stock) | |
| Solution (Prod. | | |
| No. C 149) | | |
| L-Asparagine | 800 mg/L | Phytotechnology Labs |
| Myo-inositol | 100 mg/L | Sigma |
| L-Proline | 1.4 g/L | Phytotechnology Labs |
| Casaminoacids | 100 mg/L | Fisher Scientific |
| Sucrose | 50 g/L | Phytotechnology Labs |
| 2,4-D (Prod. No. | 1 mL/L (of 1 mg/mL | Sigma |
| D-7299) | Stock) | |

Adjust the pH of the solution to pH to 5.8 with 1N KOH/1N KCl, add Gelrite (Sigma) to 3 g/L, and autoclave. After cooling to 50° C., add 2 ml/L of a 5 mg/ml stock solution of Silver Nitrate (Phytotechnology Labs). Recipe yields about 20 plates.

### Example 17

### Transformation of GDC-1 into Plant Cells by Agrobacterium-Mediated Transformation

Ears are collected 8-12 days after pollination. Embryos are isolated from the ears, and those embryos 0.8-1.5 mm in size are used for transformation. Embryos are plated scutellum side-up on a suitable incubation media, and incubated overnight at 25° C. in the dark. However, it is not necessary per se to incubate the embryos overnight. Embryos are contacted with an Agrobacterium strain containing the appropriate vectors for Ti plasmid mediated transfer for 5-10 min, and then plated onto co-cultivation media for 3 days (25° C. in the dark). After co-cultivation, explants are transferred to recovery period media for five days (at 25° C. in the dark). Explants are incubated in selection media for up to eight weeks, depending on the nature and characteristics of the particular selection utilized. After the selection period, the resulting callus is transferred to embryo maturation media, until the formation of mature somatic embryos is observed. The resulting mature somatic embryos are then placed under low light, and the process of regeneration is initiated as known in the art. The resulting shoots are allowed to root on rooting media, and the resulting plants are transferred to nursery pots and propagated as transgenic plants.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

That which is claimed:

1. An isolated nucleic acid molecule selected from the group consisting of:
a) a nucleic acid molecule comprising the nucleotide sequence of SEQ ID NO:1,2,4,5,7,9; 10, 18, or 20;
b) a nucleic acid molecule comprising a nucleotide sequence having at least 90% sequence identity to the nucleotide sequence of SEQ ID NO:1, 2, 4, 5, 7, 9, 10, 18, or 20, wherein said nucleotide sequence encodes a polypeptide having glyphosate resistance activity;
c) a nucleic acid molecule which encodes a polypeptide comprising the amino acid sequence of SEQ ID NO:3, 6, 8, 11, 19, or 21;
d) a nucleic acid molecule comprising a nucleotide sequence encoding a polypeptide having at least 90% amino acid sequence identity to the amino acid sequence of SEQ ID NO:3, 6, 8, 11, 19, or 21, wherein said polypeptide has glyphosate resistance activity; and,
e) a complement of any of a)-d).

2. An isolated nucleic acid molecule of claim 1, wherein said nucleotide sequence is a synthetic sequence that has been designed for expression in a plant.

3. The nucleic acid molecule of claim 2, wherein said synthetic sequence has an increased GC content.

4. A vector comprising the nucleic acid molecule of claim 1.

5. The vector of claim 4, further comprising a nucleic acid molecule encoding a heterologous polypeptide.

6. A host cell that contains the vector of claim 4.

7. The host cell of claim 6 that is a bacterial host cell.

8. The host cell of claim 6 that is a plant cell.

9. A transgenic plant comprising the host cell of claim 8.

10. The plant of claim 9, wherein said plant is selected from the group consisting of maize, sorghum, wheat, sunflower, tomato, crucifers, peppers, potato, cotton, rice, soybean, sugarbeet, sugarcane, tobacco, barley, and oilseed rape.

11. Transgenic seed of a plant of claim 9.

12. An isolated polypeptide selected from the group consisting of:
a) a polypeptide comprising the amino acid sequence of SEQ NO:3, 6, 8, 11, 19, or 21;
b) a polypeptide encoded by the nucleotide sequence of SEQ ID NO:1, 2, 4, 5, 7, 9, 10, 18, or 20;
c) a polypeptide comprising an amino acid sequence having at least 90% sequence identity to the amino acid sequence of SEQ ID NO:3, 6, 8, 11, 19, or 21, wherein said polypeptide has glyphosate resistance activity; and,
d) a polypeptide that is encoded by a nucleotide sequence that is at least 90% identical to the nucleotide sequence of SEQ ID NO:1, 2, 4, 5, 7, 9, 10, 18, or 20, wherein said polypeptide has glyphosate resistance activity.

13. The polypeptide of claim 12 further comprising a heterologous amino acid sequence.

14. A method for producing a polypeptide with glyphosate resistance activity, comprising culturing the host cell of claim 6 under conditions in which a nucleic acid molecule encoding the polypeptide is expressed, said polypeptide being selected from the group consisting of:
a) a polypeptide comprising the amino acid sequence of SEQ ID NO:3, 6, 8, 11, 19, or 21;
b) a polypeptide encoded by the nucleic acid sequence of SEQ ID NO:1, 2, 4, 5, 7, 9, 10, 18, or 20;
c) a polypeptide comprising an amino acid sequence having at least 90% sequence identity to a polypeptide with the amino acid sequence of SEQ ID NO:3, 6, 8, 11, 19, or 21, wherein said polypeptide has glyphosate resistance activity; and,
d) a polypeptide encoded by a nucleic acid molecule comprising a nucleotide sequence having at least 90% sequence identity to the nucleic acid sequence of SEQ ID NO:1, 2, 4, 5, 7, 9, 10, 18, or 20, wherein said polypeptide has glyphosate resistance activity.

15. A method for conferring resistance to glyphosate in a plant, said method comprising transforming said plant with a DNA construct, said construct comprising a promoter that drives expression in a plant cell operably linked with a nucleotide sequence at least 90% identical to the nucleotide sequence of SEQ ID NO:3, 6, 8, 11, 19, or 21, and regenerating a transformed plant.

16. A plant having stably incorporated into its genome a DNA construct comprising a nucleotide sequence that encodes a protein having glyphosate resistance activity, wherein said nucleotide sequence is selected from the group consisting of:
a) a nucleotide sequence of SEQ ID NO:1, 2, 4, 5, 7, 9, 10, 18, or 20;
b) a nucleotide sequence having at least 90% sequence identity to a nucleotide sequence of SEQ ID NO:1, 2, 4, 5, 7, 9, 10, 18, or 20, wherein said nucleotide sequence encodes a polypeptide having glyphosate resistance activity;
c) a nucleotide sequence encoding a polypeptide comprising an amino acid sequence of SEQ ID NO:3, 6, 8, 11, 19, or 21; and,
d) a nucleotide sequence encoding a polypeptide having at least 90% amino acid sequence identity to the amino acid sequence of SEQ ID NO:3, 6, 8, 11, 19, or 21, wherein said polypeptide has glyphosate resistance activity; wherein said nucleotide sequence is operably linked to a promoter that drives expression of a coding sequence in a plant cell.

17. A plant cell having stably incorporated into its genome a DNA construct comprising a nucleotide sequence that encodes a protein having herbicide resistance activity, wherein said nucleotide sequence is selected from the group consisting of:
a) a nucleotide sequence of SEQ ID NO:1, 2, 4, 5, 7, 9, 10, 18, or 20;
b) a nucleotide sequence having at least 90% sequence identity to a nucleotide sequence of SEQ ID NO:1, 2, 4, 5, 7, 9, 10, 18, or 20, wherein said nucleotide sequence encodes a polypeptide having glyphosate resistance activity;
c) a nucleotide sequence encoding a polypeptide comprising an amino acid sequence of SEQ ID NO:3, 6, 8, 11, 19, or 21; and,
d) a nucleotide sequence encoding a polypeptide having at least 90% amino acid sequence identity to the amino acid sequence of SEQ ID NO:3, 6, 8, 11, 19, or 21, wherein said polypeptide has glyphosate resistance activity; wherein said nucleotide sequence is operably linked to a promoter that drives expression of a coding sequence in a plant cell.


SEQUENCE LISTING (21 sequences)

SEQ ID NO: 1 - DNA, 2210 bp, Unknown
  CDS (224)...(1951): Fungal isolate from soil sample
acgcggggtgcccacggacaacaattcccttaggattatctcctgtattgaatacactctactttgcaactttacctattattcgactttcttttagaggagcagcattgtcatcattacctgcccctccatctgatacctaccttacattgtcgccaacacacctataagccataatataccgactcaaagcaaaccacgcccattgtttgattgtttaatcatggccagcatcaacatcagggtgcagaatctcgagcaacccatggacgttgccgagtatctttttcggcgtctccacgaaatcggcattcgctccatccacggtcttccaggcgattacaaccttcttgccctcgactatttgccatcatgtggcctgagatgggttggcagcgtcaacgaactcaatgctgcttatgctgctgatggctatgcccgcgtcaagcagatgggagctctcatcaccacttttggagtgggagagctctcagccatcaatggcgttgccggtgccttttcggaacacgtcccagtcgttcacattgttggctgcccttccactgtctcgcagcgaaacggcatgctcctccaccacacgcttggaaacggcgacttcaacatctttgccaacatgagcgctcaaatctcttgcgaagtggccaagctcaccaaccctgccgaaattgcgacccagatcgaccatgccctccgcgtttgcttcattcgttctcggcccgtctacatcatgcttcccaccgatatggtccaggccaaagtagaaggtgccagactcaaggaaccaattgacttgtcggagcctccaaatgatcccgagaaagaagcatacgtcgttgacgttgtcctcaagtayctccgtgctgcaaagaaccccgtcatccttgtcgatgcttgtgctatccgtcatcgtgttcttgatgaggttcatgatctcatcgaaaagacaaacctccctgtctttgtcactcctatgggcaaaggtgctgttaacgaagaacacccgacatatggtggtgtctatgccggtgacggctcacatccgcctcaagttaaggacatggttgagtcttctgatttgatattgacaatcggtgctctcaagagcgacttcaacactgctggcttctcttaccgtacctcacagctgaacacgattgatctacacagcgaccactgcattgtcaaatactcgacatatccaggtgtccagatgaggggtgtgctgcgacaagtgattaagcagctcgatgcatctgagatcaacgctcagccagcgccagtcgtcgagaatgaagttgccaaaaaccgagataactcacccgtcattacacaagctttcttctggccgcgcgtgggagagttcctgaagaagaacgacatcgtcattaccgagactggaacagccaactttggcatctgggatactaagtttccctctggcgttactgcgctttctcaggtcctttggggaagcattggttggtccgttggtgcctgccaaggagccgttcttgcagccgccgatgacaacagcgatcgcagaactatcctctttgttggtgatggctcattccagctcactgctcaagaattgagcacaatgattcgtctcaagctgaagcccatcatctttgtcatctgcaacgatggctttaccattgaacgattcattcacggcatggaagccgagtacaacgacatcgcaaattgggacttcaaggctctggttgacgtctttggcggctctaagacggccaagaagttcgccgtcaagaccaaggacgagctggacagccttctcacagaccctacctttaacgccgcagaatgcctccagtttgtcgagctatatatgcccaaagaagatgctcctcgagcattgatcatgactgcagaagctagcgcgaggaacaatgccaagacagagtaaagtggactgtcatgaaggccgatttaccacctcataaattgtaatagacctgatacacatagatcaaggcaggtaccgatcattaatcaagcaggtttggatggggaaggattttgaaaatgaggaaacgatgggatgatatttggaataactggccattattttgagtacttataaacaaatttgaagttcaattttttttcaaaaaaaaaaaaaaaaaaaaaaaaaaaaaaaaaaaaaaaaaaaaaaaaaaaaaaaa

SEQ ID NO: 2 - DNA, 1725 bp, Unknown
  CDS (1)...(1725): Fungal isolate from soil sample
atggccagcatcaacatcagggtgcagaatctcgagcaacccatggacgttgccgagtatctttttcggcgtctccacgaaatcggcattcgctccatccacggtcttccaggcgattacaaccttcttgccctcgactatttgccatcatgtggcctgagatgggttggcagcgtcaacgaactcaatgctgcttatgctgctgatggctatgcccgcgtcaagcagatgggagctctcatcaccacttttggagtgggagagctctcagccatcaatggcgttgccggtgccttttcggaacacgtcccagtcgttcacattgttggctgcccttccactgtctcgcagcgaaacggcatgctcctccaccacacgcttggaaacggcgacttcaacatctttgccaacatgagcgctcaaatctcttgcgaagtggccaagctcaccaaccctgccgaaattgcgacccagatcgaccatgccctccgcgtttgcttcattcgttctcggcccgtctacatcatgcttcccaccgatatggtccaggccaaagtagaaggtgccagactcaaggaaccaattgacttgtcggagcctccaaatgatcccgagaaagaagcatacgtcgttgacgttgtcctcaagtayctccgtgctgcaaagaaccccgtcatccttgtcgatgcttgtgctatccgtcatcgtgttcttgatgaggttcatgatctcatcgaaaagacaaacctccctgtctttgtcactcctatgggcaaaggtgctgttaacgaagaacacccgacatatggtggtgtctatgccggtgacggctcacatccgcctcaagttaaggacatggttgagtcttctgatttgatattgacaatcggtgctctcaagagcgacttcaacactgctggcttctcttaccgtacctcacagctgaacacgattgatctacacagcgaccactgcattgtcaaatactcgacatatccaggtgtccagatgaggggtgtgctgcgacaagtgattaagcagctcgatgcatctgagatcaacgctcagccagcgccagtcgtcgagaatgaagttgccaaaaaccgagataactcacccgtcattacacaagctttcttctggccgcgcgtgggagagttcctgaagaagaacgacatcgtcattaccgagactggaacagccaactttggcatctgggatactaagtttccctctggcgttactgcgctttctcaggtcctttggggaagcattggttggtccgttggtgcctgccaaggagccgttcttgcagccgccgatgacaacagcgatcgcagaactatcctctttgttggtgatggctcattccagctcactgctcaagaattgagcacaatgattcgtctcaagctgaagcccatcatctttgtcatctgcaacgatggctttaccattgaacgattcattcacggcatggaagccgagtacaacgacatcgcaaattgggacttcaaggctctggttgacgtctttggcggctctaagacggccaagaagttcgccgtcaagaccaaggacgagctggacagccttctcacagaccctacctttaacgccgcagaatgcctccagtttgtcgagctatatatgcccaaagaagatgctcctcgagcattgatcatgactgcagaagctagcgcgaggaacaatgccaagacagag

SEQ ID NO: 3 - protein, 575 aa, Unknown
  Fungal isolate from soil sample
MASINIRVQNLEQPMDVAEYLFRRLHEIGIRSIHGLPGDYNLLALDYLPSCGLRWVGSVNELNAAYAADGYARVKQMGALITTFGVGELSAINGVAGAFSEHVPVVHIVGCPSTVSQRNGMLLHHTLGNGDFNIFANMSAQISCEVAKLTNPAEIATQIDHALRVCFIRSRPVYIMLPTDMVQAKVEGARLKEPIDLSEPPNDPEKEAYVVDVVLKYLRAAKNPVILVDACAIRHRVLDEVHDLIEKTNLPVFVTPMGKGAVNEEHPTYGGVYAGDGSHPPQVKDMVESSDLILTIGALKSDFNTAGFSYRTSQLNTIDLHSDHCIVKYSTYPGVQMRGVLRQVIKQLDASEINAQPAPVVENEVAKNRDNSPVITQAFFWPRVGEFLKKNDIVITETGTANFGIWDTKFPSGVTALSQVLWGSIGWSVGACQGAVLAAADDNSDRRTILFVGDGSFQLTAQELSTMIRLKLKPIIFVICNDGFTIERFIHGMEAEYNDIANWDFKALVDVFGGSKTAKKFAVKTKDELDSLLTDPTFNAAECLQFVELYMPKEDAPRALIMTAEASARNNAKTE

SEQ ID NO: 4 - DNA, 835 bp, Unknown
  CDS (3)...(596): Fungal isolate from soil sample
ctttcttctggccgcgcgtgggagagttcctgaagaagaacgacatcgtcattaccgagactggaacagccaactttggcatctgggatactaagtttccctctggcgttactgcgctttctcaggtcctttggggaagcattggttggtccgttggtgcctgccaaggagccgttcttgcagccgccgatgacaacagcgatcgcagaactatcctctttgttggtgatggctcattccagctcactgctcaagaattgagcacaatgattcgtctcaagctgaagcccatcatctttgtcatctgcaacgatggctttaccattgaacgattcattcacggcatggaagccgagtacaacgacatcgcaaattgggacttcaaggctctggttgacgtctttggcggctctaagacggccaagaagttcgccgtcaagaccaaggacgagctggacagccttctcacagaccctacctttaacgccgcagaatgcctccagtttgtcgagctatatatgcccaaagaagatgctcctcgagcattgatcatgactgcagaagctagcgcgaggaacaatgccaagacagagtaaagtggactgtcatgaaggccgatttaccacctcataaattgtaatagacctgatacacatagatcaaggcaggtaccgatcattaatcaagcaggtttggatggggaaggattttgaaaatgaggaaacgatgggatgatatttggaataactggccattattttgagtacttataaacaaatttgaagttcaattttttttcaaaaaaaaaaaaaaaaaaaaaaaaaaaaaaaaaaaa

SEQ ID NO: 5 - DNA, 591 bp, Unknown
  CDS (1)...(591): Fungal isolate from soil sample
ttcttctggccgcgcgtgggagagttcctgaagaagaacgacatcgtcattaccgagactggaacagccaactttggcatctgggatactaagtttccctctggcgttactgcgctttctcaggtcctttggggaagcattggttggtccgttggtgcctgccaaggagccgttcttgcagccgccgatgacaacagcgatcgcagaactatcctctttgttggtgatggctcattccagctcactgctcaagaattgagcacaatgattcgtctcaagctgaagcccatcatctttgtcatctgcaacgatggctttaccattgaacgattcattcacggcatggaagccgagtacaacgacatcgcaaattgggacttcaaggctctggttgacgtctttggcggctctaagacggccaagaagttcgccgtcaagaccaaggacgagctggacagccttctcacagaccctacctttaacgccgcagaatgcctccagtttgtcgagctatatatgcccaaagaagatgctcctcgagcattgatcatgactgcagaagctagcgcgaggaacaatgccaagacagag

SEQ ID NO: 6 - protein, 197 aa, Unknown
  Fungal isolate from soil sample
FFWPRVGEFLKKNDIVITETGTANFGIWDTKFPSGVTALSQVLWGSIGWSVGACQGAVLAAADDNSDRRTILFVGDGSFQLTAQELSTMIRLKLKPIIFVICNDGFTIERFIHGMEAEYNDIANWDFKALVDVFGGSKTAKKFAVKTKDELDSLLTDPTFNAAECLQFVELYMPKEDAPRALIMTAEASARNNAKTE

SEQ ID NO: 7 - DNA, 678 bp, Unknown
  CDS (1)...(678): Fungal isolate from soil sample
acatatccaggtgtccagatgaggggtgtgctgcgacaagtgattaagcagctcgatgcatctgagatcaacgctcagccagcgccagtcgtcgagaatgaagttgccaaaaaccgagataactcacccgtcattacacaagctttcttctggccgcgcgtgggagagttcctgaagaagaacgacatcgtcattaccgagactggaacagccaactttggcatctgggatactaagtttccctctggcgttactgcgctttctcaggtcctttggggaagcattggttggtccgttggtgcctgccaaggagccgttcttgcagccgccgatgacaacagcgatcgcagaactatcctctttgttggtgatggctcattccagctcactgctcaagaattgagcacaatgattcgtctcaagctgaagcccatcatctttgtcatctgcaacgatggctttaccattgaacgattcattcacggcatggaagccgagtacaacgacatcgcaaattgggacttcaaggctctggttgacgtctttggcggctctaagacggccaagaagttcgccgtcaagaccaaggacgagctggacagccttctcacagaccctacctttaacgccgcagaatgcctccagtttgtcgagctatatatgcccaaagaagatgct

SEQ ID NO: 8 - protein, 226 aa, Unknown
  Fungal isolate from soil sample
TYPGVQMRGVLRQVIKQLDASEINAQPAPVVENEVAKNRDNSPVITQAFFWPRVGEFLKKNDIVITETGTANFGIWDTKFPSGVTALSQVLWGSIGWSVGACQGAVLAAADDNSDRRTILFVGDGSFQLTAQELSTMIRLKLKPIIFVICNDGFTIERFIHGMEAEYNDIANWDFKALVDVFGGSKTAKKFAVKTKDELDSLLTDPTFNAAECLQFVELYMPKEDA

SEQ ID NO: 9 - DNA, 1636 bp, Unknown
  CDS (1)...(1377): Fungal isolate from soil sample
cgaaacggcatgctcctccaccacacgcttggaaacggcgacttcaacatctttgccaacatgagcgctcaaatctcttgcgaagtggccaagctcaccaaccctgccgaaattgcgacccagatcgaccatgccctccgcgtttgcttcattcgttctcggcccgtctacatcatgcttcccaccgatatggtccaggccaaagtagaaggtgccagactcaaggaaccaattgacttgtcggagcctccaaatgatcccgagaaagaagcatacgtcgttgacgttgtcctcaagtacctccgtgctgcaaagaaccccgtcatccttgtcgatgcttgtgctatccgtcatcgtgttcttgatgaggttcatgatctcatcgaaaagacaaacctccctgtctttgtcactcctatgggcaaaggtgctgttaacgaagaacacccgacatatggtggtgtctatgccggtgacggctcacatccgcctcaagttaaggacatggttgagtcttctgatttgatattgacaatcggtgctctcaagagcgacttcaacactgctggcttctcttaccgtacctcacagctgaacacgattgatctacacagcgaccactgcattgtcaaatactcgacatatccaggtgtccagatgaggggtgtgctgcgacaagtgattaagcagctcgatgcatctgagatcaacgctcagccagcgccagtcgtcgagaatgaagttgccaaaaaccgagataactcacccgtcattacacaagctttcttctggccgcgcgtgggagagttcctgaagaagaacgacatcgtcattaccgagactggaacagccaactttggcatctgggatactaagtttccctctggcgttactgcgctttctcaggtcctttggggaagcattggttggtccgttggtgcctgccaaggagccgttcttgcagccgccgatgacaacagcgatcgcagaactatcctctttgttggtgatggctcattccagctcactgctcaagaattgagcacaatgattcgtctcaagctgaagcccatcatctttgtcatctgcaacgatggctttaccattgaacgattcattcacggcatggaagccgagtacaacgacatcgcaaattgggacttcaaggctctggttgacgtctttggcggctctaagacggccaagaagttcgccgtcaagaccaaggacgagctggacagccttctcacagaccctacctttaacgccgcagaatgcctccagtttgtcgagctatatatgcccaaagaagatgctcctcgagcattgatcatgactgcagaagctagcgcgaggaacaatgccaagacagagtaaagtggactgtcatgaaggccgatttaccacctcataaattgtaatagacctgatacacatagatcaaggcaggtaccgatcattaatcaagcaggtttggatggggaaggattttgaaaatgaggaaacgatgggatgatatttggaataactggccattattttgagtacttataaacaaatttgaagttcaattttttttcaaaaaaaaaaaaaaaaaaaaaaaaaaaaaaaaaaaaaaaaaaaaaaaaaaaaaaaa

SEQ ID NO: 10 - DNA, 1374 bp, Unknown
  CDS (1)...(1374): Fungal isolate from soil sample
cgaaacggcatgctcctccaccacacgcttggaaacggcgacttcaacatctttgccaacatgagcgctcaaatctcttgcgaagtggccaagctcaccaaccctgccgaaattgcgacccagatcgaccatgccctccgcgtttgcttcattcgttctcggcccgtctacatcatgcttcccaccgatatggtccaggccaaagtagaaggtgccagactcaaggaaccaattgacttgtcggagcctccaaatgatcccgagaaagaagcatacgtcgttgacgttgtcctcaagtacctccgtgctgcaaagaaccccgtcatccttgtcgatgcttgtgctatccgtcatcgtgttcttgatgaggttcatgatctcatcgaaaagacaaacctccctgtctttgtcactcctatgggcaaaggtgctgttaacgaagaacacccgacatatggtggtgtctatgccggtgacggctcacatccgcctcaagttaaggacatggttgagtcttctgatttgatattgacaatcggtgctctcaagagcgacttcaacactgctggcttctcttaccgtacctcacagctgaacacgattgatctacacagcgaccactgcattgtcaaatactcgacatatccaggtgtccagatgaggggtgtgctgcgacaagtgattaagcagctcgatgcatctgagatcaacgctcagccagcgccagtcgtcgagaatgaagttgccaaaaaccgagataactcacccgtcattacacaagctttcttctggccgcgcgtgggagagttcctgaagaagaacgacatcgtcattaccgagactggaacagccaactttggcatctgggatactaagtttccctctggcgttactgcgctttctcaggtcctttggggaagcattggttggtccgttggtgcctgccaaggagccgttcttgcagccgccgatgacaacagcgatcgcagaactatcctctttgttggtgatggctcattccagctcactgctcaagaattgagcacaatgattcgtctcaagctgaagcccatcatctttgtcatctgcaacgatggctttaccattgaacgattcattcacggcatggaagccgagtacaacgacatcgcaaattgggacttcaaggctctggttgacgtctttggcggctctaagacggccaagaagttcgccgtcaagaccaaggacgagctggacagccttctcacagaccctacctttaacgccgcagaatgcctccagtttgtcgagctatatatgcccaaagaagatgctcctcgagcattgatcatgactgcagaagctagcgcgaggaacaatgccaagacagag

SEQ ID NO: 11 - protein, 458 aa, Unknown
  Fungal isolate from soil sample
RNGMLLHHTLGNGDFNIFANMSAQISCEVAKLTNPAEIATQIDHALRVCFIRSRPVYIMLPTDMVQAKVEGARLKEPIDLSEPPNDPEKEAYVVDVVLKYLRAAKNPVILVDACAIRHRVLDEVHDLIEKTNLPVFVTPMGKGAVNEEHPTYGGVYAGDGSHPPQVKDMVESSDLILTIGALKSDFNTAGFSYRTSQLNTIDLHSDHCIVKYSTYPGVQMRGVLRQVIKQLDASEINAQPAPVVENEVAKNRDNSPVITQAFFWPRVGEFLKKNDIVITETGTANFGIWDTKFPSGVTALSQVLWGSIGWSVGACQGAVLAAADDNSDRRTILFVGDGSFQLTAQELSTMIRLKLKPIIFVICNDGFTIERFIHGMEAEYNDIANWDFKALVDVFGGSKTAKKFAVKTKDELDSLLTDPTFNAAECLQFVELYMPKEDAPRALIMTAEASARNNAKTE

SEQ ID NO: 12 - DNA, 30 bp, Unknown
  CDS (1)...(30): Oligonucleotide used for PCR amplification of GDC-1
tcccagatgccaaagttggctgttccagtc

SEQ ID NO: 13 - protein, 563 aa, Saccharomyces cerevisiae
MSEITLGKYLFERLKQVNVNTVFGLPGDFNLSLLDKIYEVEGMRWAGNANELNARYAADGYARIKGMSCIITTFGVGELSALNGIAGSYAEHVGVLHVVGVPSISSQAKQLLLHHTLGNGDFTVFHRMSANISETTAMITDICTAPAEIDRCIRTTYVTQRPVYLGLPANLVDLNVPAKLLQTPIDMSLKPNDAESEKEVIDTILVLAKDAKNPVILADACCSRHDVKAETKKLIDLTQFPAFVTPMGKGSISEQHPRYGGVYVGTLSKPEVKEAVESADLILSVGALLSDFNTGSFSYSYKTKNIVEFHSDHMKIRNATFPGVQMKFVLQKLLTNIADAAKGYKPVAVPARTPANAAVPASTPLKQEWMWNQLGNFLQEGDVVIAETGTSAFGINQTTFPNNTYGISQVLWGSIGFTTGATLGAAFAAEEIDPKKRVILFIGDGSLQLTVQEISTMIRWGLKPYLFVLNNDGYTIEKLIHGPKAQYNEIQGWDHLSLLPTFGAKDYETHRVATTGEWDKLTQDKSFNDNSKIRMIEVMLPVFDAPQNLVEQAKLTAATNAKQ

SEQ ID NO: 14 - protein, 550 aa, Salmonella typhimurium
MQNPYTVADYLLDRLAGCGIGHLFGVPGDYNLQFLDHVIDHPTLRWVGCANELNAAYAADGYARMSGAGALLTTFGVGELSAINGIAGSYAEYVPVLHIVGAPCSAAQQRGELMHHTLGDGDFRHFYRMSQAISAASAILDEQNACFEIDRVLGEMLAARRPGYIMLPADVAKKTAIPPTQALALPVHEAQSGVETAFRYHARQCLMNSRRIALLADFLAGRFGLRPLLQRWMAETPIAHATLLMGKGLFDEQHPNFVGTYSAGASSKEVRQAIEDADRVICVGTRFVDTLTAGFTQQLPAERTLEIQPYASRIGETWFNLPMAQAVSTLRELCLECAFAPPPTRSAGQPVRIDKGELTQESFWQTLQQYLKPGDIILVDQGTAAFGAAALSLPDGAEVVLQPLWGSIGYSLPAAFGAQTACPDRRVILIIGDGAAQLTIQEMGSMLRDGQAPVILLLNNDGYTVERAIHGAAQRYNDIASWNWTQIPPALNAAQQAECWRVTQAIQLAEVLERLARPQRLSFIEVMLPKADLPELLRTVTRALEARNGG

SEQ ID NO: 15 - protein, 568 aa, Zymomonas mobilis
MSYTVGTYLAERLVQIGLKHHFAVAGDYNLVLLDNLLLNKNMEQVYCCNELNCGFSAEGYARAKGAAAAVVTYSVGAHSAFDAIGGAYAENLPVILISGAPNNNDHAAGHVLHHALGKTDYHYQLEMAKNITAAAEAIYTPEEAPAKIDHVIKTALAKKKPVYLEIACNIASMPCAAPGPASALFNDEASDEASLNAAVDETLKFIANRDKVAVLVGSKLRAAGAEEAAVKFTDALGGAVATMAAAKSFFPEENPHYIGTSWGEVSYPGVEKTMKEADAVIALAPVFNDYSTTGWTDIPDPKKLVLAEPRSVVVRRIRFPSVHLKDYLTRLAQKVSKKTGSLDFFKSLNAGELKKAAPADPSAPLVNAEIARQVEALLTPNTTVIAETGDSWFNAQRMKLPNGARVEYEMQWGHIGWSVPAAFGYAVGAPERRNILMVGDGSFQLTAQEVAQMVRLKLPVIIFLINNYGYTIEVMIHDGPYNNIKNWDYAGLMEVFNGNGGYDSGAAKGLKAKTGGELAEAIKVALANTDGPTLIECFIGREDCTEELVKWGKRVAAANSRKPVNKLL

SEQ ID NO: 16 - protein, 687 aa, Saccharomyces cerevisiae [sequence omitted]

SEQ ID NO: 17 - protein, 686 aa, Magnaporthe grisea
MLRTVGRKALRGSSKGCSRTISTLKPATATIAKPGSRTLSTPATATATAPRTKPSASFNARRDPQPLVNPRSGEADESFIGKTGGEIFHEMMLRQNVKHIFGYPGGAILPVFDAIYNSKHIDFVLPKHEQGAGHMAEGYARASGKPGVVLVTSGPGATNVITPMADALADGTPLVVFSGQVVTSDIGSDAFQEADVIGISRSCTKWNVMVKSADELPRRINEAFEIATSGRPGPVLVDPAKDVTASVLRRAIPTETSIPSISAAARAVQEAGRKQLEHSIKRVADLVNIAKKPVIYAGQGVILSEGGVELLKALADKASIPVTTTLHGLGAFDELDEKALHMLGMHGSAYANMSMQEADLIIALGGRFDDRVTGSIPKFAPAAKLAAAEGRGGIVHFEIMPKNINKVVQATEAIEGDVASNLKLLLPKIEQRSMTDRKEWFDQIKEWKEKWPLSHYERAERSGLIKPQTLIEELSNLTADRKDMTYITTGVGQHQMWTAQHFRWRHPRSMITSGGLGTMGYGLPAAIGAKVARPDALVIDIDGDASFNMTLTELSTAAQFNIGVKVIVLNNEEQGMVTQWQNLFYEDRYSHTHQRNPDFMKLADAMDVQHRRVSKPDDVGDALTWLINTDGPALLEVMTDKKVPVLPMVPGGNGLHEFITFDASKDKQRRELMRARTNGLHGRTAV

SEQ ID NO: 18 - DNA, 1728 bp, Unknown
  Fungal isolate from soil sample
atggccagcatcaacatcagggtgcagaatctcgagcaacccatggacgttgccgagtatcttttccggcgtctccacgaaatcggcattcgctccatccacggtcttccaggcgattacaaccctcttgccctcgactatttgccatcatgtggcctgagatgggttggcagcgtcaacgaactcaatgctgcttatgctgctgatggctatgcccgcgtcaagcagatgggagctctcatcaccacttttggagtgggagagctctcagccatcaatggcgttgccggtgccttttcggaacacgtcccagtcgttcacattgttggctgcccttccactgcctcgcagcgaaacggcatgctcctccaccacacgcttggaaacggcgacttcaacatctttgccaacatgagcgctcaaatctcttgcgaagtggccaagctcaccaaccctgccgaaattgcgacccagatcgaccatgccctccgcgtttgcttcattcgttctcggcccgtctacatcatgcttcccaccgatatggtccaggccaaagtagaaggtgccagactcaaggaaccaattgacttgtcggagcctccaaatgatcccgagaaagaagcatacgtcgttgacgttgtcctcaagtacctccgtgctgcaaagaaccccgtcatccttgtcgatgcttgtgctatccgtcatcgtgttcttgatgaggttcatgatctcatcgaaaagacaaacctccccgtctttgtcactcctatgggcaaaggtgctgttaacgaagaacacccgacatatggtggtgtctatgccggtgacggctcacatccgcctcaagttaaggacatggttgagtcttctgatttgatattgacaatcggtgctctcaagagcgacttcaacactgctggcttctcttaccgtacctcacagctgaacacgattgatctacacagcgaccactgcattgtcaaatactcgacatatccaggtgtccagatgaggggtgtgctgcgacaagtgattaagcagctcgatgcatctgagatcaacgctcagccagcgccagtcgtcgagaatgaagttgccaaaaaccgagataactcacccgtcattacacaagctttcttctggccgcgcgtgggagagttcctgaagaagaacgacatcgtcattaccgagactggaacagccaactttggcatctgggatactaagtttccctctggcgttactgcgctttctcaggtcctttggggaagcattggttggtccgttggtgcctgccaaggagccgttcttgcagccgccgatgacaacagcgatcgcagaactatcctctttgttggtgatggctcattccagctcactgctcaagaattgagcacaatgattcgtctcaagctgaagcccatcatctttgtcatctgcaacgatggctttaccattgaacgattcattcacggcatggaagccgagtacaacgacatcgcaaattgggacttcaaggctctggttgacgtctttggcggctctaagacggccaagaagttcgccgtcaagaccaaggacgagctggacagccttctcacagaccctacctttaacgccgcagaatgcctccagtttgtcgagctatatatgcccaaagaagatgctcctcgagcattgatcatgacggcagaagctagcgcgaggaacaatgccaagacagagtaa

SEQ ID NO: 19 - protein, 575 aa, Unknown
  Fungal isolate from soil sample
MASINIRVQNLEQPMDVAEYLFRRLHEIGIRSIHGLPGDYNPLALDYLPSCGLRWVGSVNELNAAYAADGYARVKQMGALITTFGVGELSAINGVAGAFSEHVPVVHIVGCPSTASQRNGMLLHHTLGNGDFNIFANMSAQISCEVAKLTNPAEIATQIDHALRVCFIRSRPVYIMLPTDMVQAKVEGARLKEPIDLSEPPNDPEKEAYVVDVVLKYLRAAKNPVILVDACAIRHRVLDEVHDLIEKTNLPVFVTPMGKGAVNEEHPTYGGVYAGDGSHPPQVKDMVESSDLILTIGALKSDFNTAGFSYRTSQLNTIDLHSDHCIVKYSTYPGVQMRGVLRQVIKQLDASEINAQPAPVVENEVAKNRDNSPVITQAFFWPRVGEFLKKNDIVITETGTANFGIWDTKFPSGVTALSQVLWGSIGWSVGACQGAVLAAADDNSDRRTILFVGDGSFQLTAQELSTMIRLKLKPIIFVICNDGFTIERFIHGMEAEYNDIANWDFKALVDVFGGSKTAKKFAVKTKDELDSLLTDPTFNAAECLQFVELYMPKEDAPRALIMTAEASARNNAKTE

SEQ ID NO: 20 - DNA, 1728 bp, Unknown
  Fungal isolate from soil sample
atggccagcatcaacatcagggtgcagaatctcgagcaacccatggacgttgccgagtatcttttccggcgtctccacgaaatcggcattcgctccatccacggtcttccaggcgattacaaccttcttgccctcgactatttgccatcatgtggcctgagatgggttggcagcgtcaacgaactcaatgctgcttatgctgctgatggctatgcccgcgtcaagcagatgggagctctcatcaccacttttggagtgggagagctctcagccatcaatggcgttgccggtgccttttcggaacacgtcccagtcgttcacattgttggctgcccttccactgcctcgcagcgaaacggcatgctcctccaccacacgcttggaaacggcgacttcaacatctttgccaacatgagcgctcaaatctcttgcgaagtggccaagctcaccaaccctgccgaaattgcgacccagatcgaccatgccctccgcgtttgcttcattcgttctcggcccgtctacatcatgcttcccaccgatatggtccaggccaaagtagaaggtgccagactcaaggaaccaattgacttgtcggagcctccaaatgatcccgagaaagaagcatacgtcgttgacgttgtcctcaagtacctccgtgctgcaaagaaccccgtcatccttgtcgatgcttgtgctatccgtcatcgtgttcttgatgaggttcatgatctcatcgaaaagacaaacctccccgtctttgtcactcctatgggcaaaggtgctgttaacgaagaacacccgacatatggtggtgtctatgccggtgacggctcacatccgcctcaagttaaggacatggttgagtcttctgatttgatattgacaatcggtgctctcaagagcgacttcaacactgctggcttctcttaccgtacctcacagctgaacacgattgatctacacagcgaccactgcattgtcaaatactcgacatatccaggtgtccagatgaggggtgtgctgcgacaagtgattaagcagctcgatgcatctgagatcaacgctcagccagcgccagtcgtcgagaatgaagttgccaaaaaccgagataactcacccgtcattacacaagctttcttctggccgcgcgtgggagagttcctgaagaagaacgacatcgtcattaccgagactggaacagccaactttggcatctgggatactaagtttccctctggcgttactgcgctttctcaggtcctttggggaagcattggttggtccgttggtgcctgccaaggagccgttcttgcagccgccgatgacaacagcgatcgcagaactatcctctttgttggtgatggctcattccagctcactgctcaagaattgagcacaatgattcgtctcaagctgaagcccatcatctttgtcatctgcaacgatggctttaccattgaacgattcattcacggcatggaagccgagtacaacgacatcgcaaattgggacttcaaggctctggttgacgtctttggcggctctaagacggccaagaagttcgccgtcaagaccaaggacgagctggacagccttctcacagaccctacctttaacgccgcagaatgcctccagtttgtcgagctatatatgcccaaagaagatgctcctcgagcattgatcatgacggcagaagctagcgcgaggaacaatgccaagacagagtaa

SEQ ID NO: 21 - protein, 575 aa, Unknown
  Fungal isolate from soil sample
MASINIRVQNLEQPMDVAEYLFRRLHEIGIRSIHGLPGDYNLLALDYLPSCGLRWVGSVNELNAAYAADGYARVKQMGALITTFGVGELSAINGVAGAFSEHVPVVHIVGCPSTASQRNGMLLHHTLGNGDFNIFANMSAQISCEVAKLTNPAEIATQIDHALRVCFIRSRPVYIMLPTDMVQAKVEGARLKEPIDLSEPPNDPEKEAYVVDVVLKYLRAAKNPVILVDACAIRHRVLDEVHDLIEKTNLPVFVTPMGKGAVNEEHPTYGGVYAGDGSHPPQVKDMVESSDLILTIGALKSDFNTAGFSYRTSQLNTIDLHSDHCIVKYSTYPGVQMRGVLRQVIKQLDASEINAQPAPVVENEVAKNRDNSPVITQAFFWPRVGEFLKKNDIVITETGTANFGIWDTKFPSGVTALSQVLWGSIGWSVGACQGAVLAAADDNSDRRTILFVGDGSFQLTAQELSTMIRLKLKPIIFVICNDGFTIERFIHGMEAEYNDIANWDFKALVDVFGGSKTAKKFAVKTKDELDSLLTDPTFNAAECLQFVELYMPKEDAPRALIMTAEASARNNAKTE